(19) United States
(12) Patent Application Publication
(10) Pub. No.: US 2018/0110415 A1
(43) Pub. Date: Apr. 26, 2018

(54) LIVING BODY MONITORING SYSTEM, PORTABLE ELECTRONIC APPARATUS, LIVING BODY MONITORING PROGRAM, COMPUTER READABLE RECORDING MEDIUM, AND LIVING BODY MONITORING METHOD

(71) Applicant: SEIKO EPSON CORPORATION, Tokyo (JP)

(72) Inventors: Hideo SASAHARA, Chino-shi (JP); Kyohei ISOHATA, Matsumoto-shi (JP)

(73) Assignee: SEIKO EPSON CORPORATION, Tokyo (JP)

(21) Appl. No.: 15/718,959

(22) Filed: Sep. 28, 2017

(30) Foreign Application Priority Data
Oct. 26, 2016 (JP) 2016-209517

(51) Int. Cl. A61B 5/01; A61B 5/00; A63B 71/06; G09B 19/00; G09B 5/02; A63B 24/00

(52) U.S. Cl. CPC A61B 5/01; A63B 2220/836; A61B 5/0008; A61B 5/742; A63B 71/0622; G09B 19/0092; G09B 5/02; A63B 24/0062; A63B 2071/0666; A63B 2230/505; A63B 2220/12; A63B 2220/89; A63B 2220/74; A63B 2220/40; A63B 2230/04; A61B 5/486

(57) ABSTRACT

A living body monitoring system includes a measurement unit that measures a deep body temperature of a user's body, a comparison unit that compares the deep body temperature with a predetermined comparison reference, a generation unit that generates information regarding a physical condition of the user on the basis of a result of the comparison, and a display that presents the information to the user.

### CROSS-REFERENCES TO RELATED APPLICATIONS

This application claims priority to Japanese Patent Application No. 2016-209517, filed Oct. 26, 2016, the entirety of which is herein incorporated by reference.

### BACKGROUND

### 1. Technical Field

The present invention relates to a living body monitoring system, a portable electronic apparatus, a living body monitoring program, a computer readable recording medium, and a living body monitoring method.

### 2. Related Art

In recent years, a portable global positioning system (GPS) electronic apparatus which can measure a user's exercise performance and can perform wireless communication has been developed (refer to JP-T-2012-524638). A user of this GPS electronic apparatus may objectively understand performance of the user and reflect the performance in a training plan. On the other hand, a technique of determining a body composition by detecting a deep body temperature of the body with a sensor is also well known (refer to JP-T-2013-509205).

However, even if users having the same physical strength perform the same training, training effects may greatly different from each other depending on the users' physical condition. For example, it is empirically known that a training effect is high if warming up is performed before starting main training. On the other hand, even if a general user can understand a deep body temperature of the user's body, it is difficult for the user to determine the influence which the deep body temperature has on the user's life or training.

### SUMMARY

An advantage of some aspects of the invention is to provide a living body monitoring system, a portable electronic apparatus, a living body monitoring program, a computer readable recording medium, and a living body monitoring method, capable of assisting a user in effectively using a deep body temperature measurement technique for the life or training of the user.

The invention can be implemented as the following forms or application examples.

### Application Example 1

A living body monitoring system according to this application example includes a measurement unit that measures a deep body temperature of a user's body, a comparison unit that compares the deep body temperature with a predetermined comparison reference, a generation unit that generates information regarding a physical condition of the user on the basis of a result of the comparison, and a display that presents the information to the user.

The measurement unit measures a deep body temperature of the user's body, the comparison unit compares the deep body temperature with a predetermined comparison reference, and the generation unit generates information regarding a physical condition of the user on the basis of a result of the comparison. The information is information based on a result of comparison with a predetermined comparison reference, and can thus be more effectively used for the life or training of a user than information itself indicating a deep body temperature. This is because an action to be performed by a user completely differs depending on a range in which a deep body temperature is included. Therefore, for example, the user may use the information as a reference when determining whether or not the content of the user's life is reconsidered or whether or not the content of the user's training is reconsidered.

### Application Example 2

In the living body monitoring system according to the application example, the information regarding the physical condition may include advice to the user.

In a case where the information includes the advice, for example, even if the user's bodily functions are reduced, and thus the user's judgment ability deteriorates, it is possible to prompt the user to perform a right action.

### Application Example 3

In the living body monitoring system according to the application example, the advice may include at least one of advice related to whether or not starting of an exercise is possible and advice related to whether or not an exercise is required to be stopped.

Therefore, it is possible to prompt the user to start or stop an exercise at a right timing corresponding to a physical condition of the user.

### Application Example 4

In the living body monitoring system according to the application example, the advice may include at least one of advice related to the necessity of water intake, advice related to the necessity of rest, advice related to the necessity of atmospheric temperature adjustment, advice related to the necessity of body temperature adjustment, and advice related to the necessity of doctor's examination.

Therefore, it is possible to recommend, to the user, any one of water intake, rest, atmospheric temperature adjustment, body temperature adjustment, and doctor's examination at right timing corresponding to a physical condition of the user.

### Application Example 5

In the living body monitoring system according to the application example, the information may include at least one of information regarding a point where water intake is possible, information regarding a point where a rest is possible, information regarding a point where atmospheric temperature adjustment is possible, information regarding a point where body temperature adjustment is possible, and information regarding a point where a doctor's examination is possible.

Therefore, it is possible to notify the user of a point where the user takes any one of water intake, a rest, atmospheric temperature adjustment, body temperature adjustment, and a doctor's examination, at a right timing corresponding to a physical condition of the user.

### Application Example 6

In the living body monitoring system according to the application example, the information may be information which can be recognized by the user through at least one of an auditory sense, a visual sense, and a tactile sense.

Therefore, the information can be recognized by the user through at least one of the user's auditory sense, visual sense, and tactile sense.

### Application Example 7

In the living body monitoring system according to the application example, the information is displayed by using at least one of a color, text, a symbol, a scale, and a pattern which can be recognized through a visual sense by the user.

Therefore, the information can be recognized by the user by using at least one of a color, text, a symbol, a scale, and a pattern.

### Application Example 8

The living body monitoring system according to the application example may further include a portable electronic apparatus which can be carried by the user and an information terminal which can be operated by other users, and the measurement unit may be mounted on the electronic apparatus, and the display may be mounted on the information terminal.

### Application Example 9

A portable electronic apparatus according to this application example includes a measurement unit that measures a deep body temperature of a user's body; a comparison unit that compares the deep body temperature with a predetermined comparison reference; a generation unit that generates information regarding a physical condition of the user on the basis of a result of the comparison; and a display that presents the information to the user.

### Application Example 10

A living body monitoring program according to this application example causes a computer to execute measuring a deep body temperature of a user's body; comparing the deep body temperature with a predetermined comparison reference; generating information regarding a physical condition of the user on the basis of a result of the comparison; and presenting the information to the user.

### Application Example 11

A computer readable recording medium according to this application example records a living body monitoring program causing a computer to execute measuring a deep body temperature of a user's body; comparing the deep body temperature with a predetermined comparison reference; generating information regarding a physical condition of the user on the basis of a result of the comparison; and presenting the information to the user.

### Application Example 12

A living body monitoring method according to this application example includes measuring a deep body temperature of a user's body; comparing the deep body temperature with a predetermined comparison reference; generating information regarding a physical condition of the user on the basis of a result of the comparison; and presenting the information to the user.

### Application Example 13

A portable electronic apparatus according to this application example measures a deep body temperature of a user's body, compares the deep body temperature with a predetermined comparison reference, generates information regarding a physical condition of the user on the basis of a result of the comparison, and presents the information to the user.

The information is information based on a result of comparison with a predetermined comparison reference, and can thus be more effectively used for the life or training of a user than information itself indicating a deep body temperature. This is because an action to be performed by a user completely differs depending on a range in which a deep body temperature is included. Therefore, for example, the user may use the information as a reference when determining whether or not the content of the user's life is reconsidered or whether or not the content of the user's training is reconsidered.

### BRIEF DESCRIPTION OF THE DRAWINGS

The invention will be described with reference to the accompanying drawings, wherein like numbers reference like elements.

FIG. 1 illustrates an example of a diagram for explaining a summary of a system.

FIG. 2 illustrates an example of a functional block diagram of a sensor device and an electronic apparatus.

FIG. 3 illustrates an example of a functional block diagram of an information terminal and a server.

FIG. 4 illustrates an example of mounting the sensor device and the electronic apparatus.

FIG. 5 illustrates examples of display screens of an HMD.

FIG. 6 illustrates an example of an exterior of a band type sensor device.

FIG. 7 illustrates an example of an exterior of a seal type sensor device.

FIG. 8 illustrates examples of display screens of the electronic apparatus in a daily life mode.

FIG. 9 illustrates examples of display screens of the electronic apparatus in a training mode.

FIG. 10 illustrates an example of a graph indicating a temporal change in a deep body temperature in the daily life mode.

FIG. 11 illustrates an example of a graph indicating a temporal change in a deep body temperature in the training mode.

FIG. 12 illustrates an example of a flowchart of a process in the daily life mode.

FIG. 13 illustrates an example of a flowchart of a starting process in the training mode.

FIG. 14 illustrates an example of a flowchart of a stopping process in the training mode.

FIG. 15 illustrates an example of a flowchart of a resuming process in the training mode.

FIG. 16 illustrates an example of a head mounted display.

### DESCRIPTION OF EXEMPLARY EMBODIMENTS

Hereinafter, a preferred embodiment of the invention will be described with reference to the drawings. The embodiments described below are not intended to improperly limit the content of the invention disclosed in the appended claims. In addition, all constituent elements described below are not essential constituent elements of the invention.

### 1. System

### 1-1. Summary of System

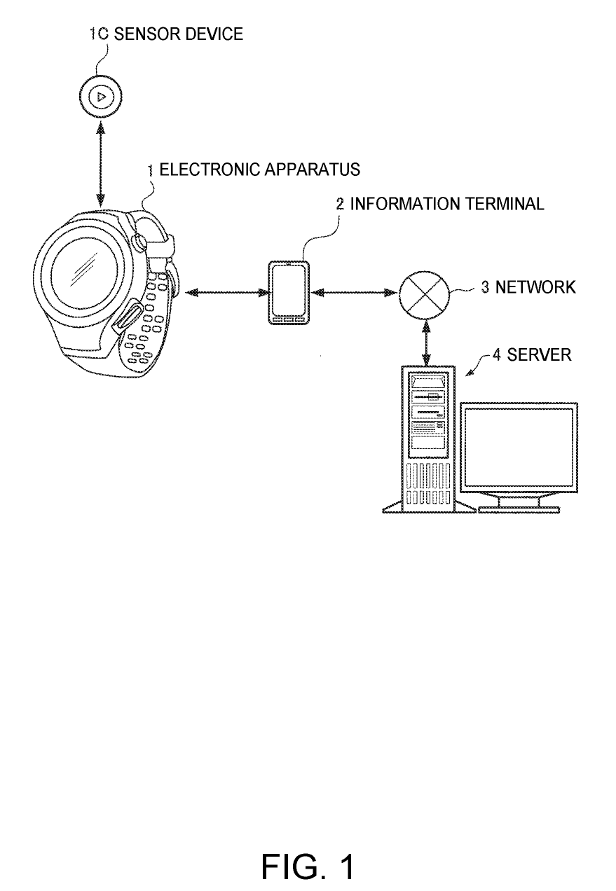

As illustrated in FIG. 1, a system (an example of a living body monitoring system) of the present embodiment includes an electronic apparatus 1 (an example of a portable electronic apparatus which can be carried by a user), a sensor device 1C, an information terminal 2, and a server 4. Each of the information terminal 2 and the server 4 is connectable to a network 3 such as the Internet, and the information terminal 2 and the server 4 can perform communication with each other via the network 3. The electronic apparatus 1 can perform communication with the information terminal 2 via short-range radio communication or the like. The sensor device 1C can perform communication with the electronic apparatus 1 via short-range radio communication or the like. The sensor device 1C and the information terminal 2 may directly communicate with each other via short-range radio communication or the like (a user may use the electronic apparatus 1 as a master device, and may use the sensor device 1C as a slave device). In the system of the present embodiment, some of the electronic apparatus 1, the sensor device 1C, the information terminal 2, and the server 4 may be omitted.

Each of the electronic apparatus 1 and the sensor device 1C is, for example, a waterproof type apparatus on which various sensors are mounted, and acquires measured data during life or training by driving at least one sensor. At least one of the electronic apparatus 1 and the sensor device 1C is mounted on a user's body during the life or training. The “training” mentioned here includes apace run, an interval run, a build-up run, long slow distance (LSD), cross country, jogging, walking, and the like, and a training location is, for example, a track or a public road.

A mounting location of the electronic apparatus 1 is, for example, a part (forearm) from the elbow to the hand of the user. The electronic apparatus 1 is formed of a wrist type (wristwatch type) electronic apparatus (outdoor watch) so that measured data regarding a user's living body can be acquired in a contact or noncontact manner, or the user can view the electronic apparatus 1 or the sensor device 1C when necessary, and a mounting location of the electronic apparatus 1 is the wrist of the user. A mounting tool (belt) suitable for a shape of the wrist is used when the electronic apparatus 1 is mounted on the wrist.

A mounting location of the sensor device 1C may be appropriately selected by the user according to the purpose of training or the like. A mounting location of the sensor device 1C is, for example, any one of the user's head, upper arm, forearm, waist, chest, thigh, crus, and ankle. Here, it is assumed that the number of sensor device 1C is one, but may be two or more. In this case, for example, the two sensor devices 1C may be separately mounted on the right wrist and the left wrist of the user. When the sensor device 1C is mounted on apart of the body, amounting tool (a belt or a clip) suitable for a shape of the part or a shape of a training wear may be used.

The information terminal 2 is an information terminal used by the user of the electronic apparatus 1 or the sensor device 1C, and is formed of, for example, a smart phone, a portable or desktop personal computer (PC) or a tablet PC. The information terminal 2 is used for the user to perform settings on the electronic apparatus 1 or the sensor device 1C, for example, before using the electronic apparatus 1 or the sensor device 1C. The information terminal 2 is used for the user to read measured data regarding the user from the electronic apparatus 1 or the sensor device 1C, for example, after using the electronic apparatus 1 or the sensor device 1C, or to upload the read measured data to the server 4. A user of the information terminal 2 may be a manager observing the life of a user wearing the electronic apparatus 1 or the sensor device 1C, or may be a coach coaching training of a user wearing the electronic apparatus 1 or the sensor device 1C.

The server 4 provides information related to use of the electronic apparatus 1 to a user of the electronic apparatus 1 or the sensor device 1C, or manages measured data acquired by the electronic apparatus 1 or the sensor device 1C for each user. The information provided by the server 4 includes at least one of a program (an example of a living body monitoring program) for operating the information terminal 2, a program for operating the electronic apparatus 1, and a program (an example of a living body monitoring program) for operating the sensor device 1C. The program of each apparatus may be downloaded to each apparatus via the network 3, and may be downloaded to each apparatus via a recording medium. This is also the same for a program (an example of a living body monitoring program) of the server 4. A method of a process performed according to the program is an example of a living body monitoring method.

### 1-2. Configuration of System

### 1-2-1. Configuration of Sensor Device

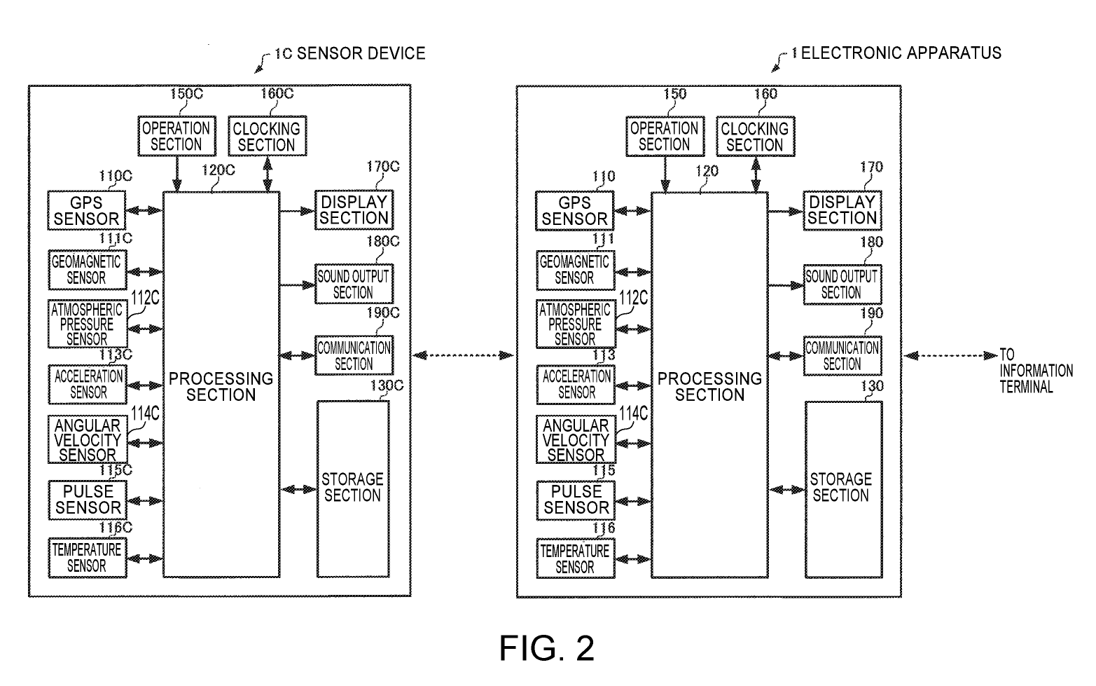

As illustrated in FIG. 2, the sensor device 1C is configured to include a GPS sensor 110C, a geomagnetic sensor 111C, an atmospheric pressure sensor 112C, an acceleration sensor 113C, an angular velocity sensor 114C, a pulse sensor 115C, a temperature sensor 116C, a processing section 120C, a storage section 130C, an operation section 150C, a clocking section 160C, a display section 170C, a sound output section 180C, a communication section 190C, and the like. However, a configuration of the sensor device 1C may have a configuration in which some of the constituent elements are deleted or changed, or may have a configuration in which other constituent elements (for example, a humidity sensor and an ultraviolet sensor) are added thereto.

The GPS sensor 110C is a sensor which generates positioning data (data such as latitude, longitude, altitude, and velocity vector) indicating a position and the like of the sensor device 1C and outputs the data to the processing section 120C, and is formed of, for example, a global positioning system (GPS) receiver. The GPS sensor 110C receives an electric wave including a satellite signal with a predetermined frequency bandwidth incoming from the outside by using a GPS antenna (not illustrated), extracts a GPS signal sent from a GPS satellite therefrom, and also generates positioning data indicating a position or the like of the sensor device 1C on the basis of the GPS signal.

The geomagnetic sensor 111C is a sensor which detects a geomagnetic vector indicating a direction of a magnetic field of the earth, viewed from the sensor device 1C, and generates, for example, geomagnetic data indicating magnetic flux densities in three-axis directions which are orthogonal to each other. For example, a magnet resistive (MR) element, a magnet impedance (MI) element, or a hole element is used for the geomagnetic sensor 111C.

The atmospheric pressure sensor 112C is a sensor which detects an ambient atmospheric pressure (atmospheric pressure), and includes, for example, a pressure sensitive element of a type (vibration type) using a change in a resonance frequency of a vibrator element. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a surface acoustic wave (SAW) vibrator. An output from the atmospheric pressure sensor 112C may be used to correct positioning data. The atmospheric pressure sensor 112C may be a MEMS type atmospheric pressure sensor manufactured by using, for example, a semiconductor manufacturing technique. Specifically, the atmospheric pressure sensor 112C includes a diaphragm portion which is bent and deformed by received pressure, and a distortion detection element which detects bending of the diaphragm portion. The diaphragm portion is made of, for example, silicon. The distortion detection element is, for example, a piezoelectric resistive element.

The acceleration sensor 113C is an inertial sensor which detects respective accelerations in the three-axis directions which intersect (are ideally orthogonal to) each other, and outputs digital signals (acceleration data) corresponding to magnitudes and directions of the detected three-axis accelerations. The outputs from the acceleration sensor 113C may be used to correct position information included in the positioning data from the GPS sensor 110C.

The angular velocity sensor 114C is an inertial sensor which detects respective angular velocities in the three-axis directions which intersect (are ideally orthogonal to) each other, and outputs digital signals (angular velocity data) corresponding to magnitudes and directions of the measured three-axis angular velocities. The outputs from the angular velocity sensor 114C may be used to correct position information included in the positioning data from the GPS sensor 110C.

The pulse sensor 115C is a sensor which generates a signal indicating a pulse of the user and outputs the signal to the processing section 120C, and includes, for example, a light source such as an LED light source which applies measurement light having an appropriate wavelength toward a blood vessel under the skin, and a light receiving element which detects a change in the intensity of light generated at the blood vessel according to the measurement light.

The temperature sensor 116C is a deep body temperature sensor which detects a deep body temperature of a user's body. The temperature sensor 116C includes, for example, a base material, a first temperature sensor which measures the temperature of a first position in the base material as a first temperature, a second temperature sensor which measures the temperature of a second position which is different from the first position, in the base material as a second temperature, a temperature sensor which measures the temperature of an outer shell body surrounding the base material as a medium temperature of the outer shell body inside, and a calculation portion which calculates a deep body temperature of a subject by using the first temperature, the second temperature, and the medium temperature. The temperature sensor 116C calculates a constant which correlates the medium temperature, the first temperature, and the second temperature with each other on the basis of the first temperature (Tb1) and the second temperature (Tp1) when the medium temperature is a first medium temperature (Tout1), the first temperature (Tb2) and the second temperature (Tp2) when the medium temperature is a second medium temperature (Tout2), and the first temperature (Tb3) and the second temperature (Tp3) when the medium temperature is a third medium temperature (Tout3), and calculates the deep body temperature (Tc). Each of such temperature sensors is, for example, a temperature sensitive element which outputs a signal (for example, a voltage corresponding to a temperature) corresponding to an ambient temperature. The temperature sensor may output a digital signal corresponding to a temperature. The temperature sensor has a pressure sensitive element of a type (vibration type) using a change in a resonance frequency of a vibrating element corresponding to an ambient temperature. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a SAW vibrator. Alternatively, the temperature sensor may be formed of a temperature sensitive element which detects a temperature with a thermocouple or a thermistor.

The storage section 130C is formed of, for example, various integrated circuit (IC) memories such as a read only memory (ROM), a flash ROM, and a random access memory (RAM), or a computer readable recording medium such as a hard disk or a memory card. The storage section 130C is formed of, for example, one or a plurality of IC memories, and includes a ROM storing data such as a program, and a RAM serving as a work region of the processing section 120C. The RAM may include a nonvolatile RAM.

The operation section 150C is formed of, for example, a button, a key, a microphone, and a touch panel so as to have a voice recognition function (using the microphone (not illustrated)) and an action detection function (using the acceleration sensor 113C or the like), and performs a process of converting an instruction from a user into an appropriate signal which is then sent to the processing section 120C.

The clocking section 160C is formed of, for example, a real time clock (RTC) IC or the like, and generates time data such as year, month, day, hour, minute, and second, and sends the data to the processing section 120C.

The display section 170C is formed of, for example, a liquid crystal display (LCD), an organic electroluminescence (EL) display, an electrophoretic display (EPD), or a touch panel display, and displays various images in response to instructions from the processing section 120C.

The sound output section 180C is formed of, for example, a speaker, a buzzer, or a vibrator, and generates various sounds (or vibration) in response to instructions from the processing section 120C.

The communication section 190C performs a variety of control for establishing data communication between the sensor device 1C and the electronic apparatus 1 (or the information terminal 2). The communication section 190C is configured to include a transceiver based on a short-range wireless communication standard such as Bluetooth (registered trademark) (including Bluetooth Low Energy (BTLE)), Wi-Fi (registered trademark) (Wireless Fidelity), Zigbee (registered trademark), near field communication (NFC), or ANT+(registered trademark).

The processing section 120C is formed of, for example, a micro processing unit (MPU), a digital signal processor (DSP), and an application specific integrated circuit (ASIC). The processing section 120C performs various processes according to a program stored in the storage section 130C, and various commands which are input by the user via the operation section 150C. The processes in the processing section 120C include, for example, data processing on data which is generated by the GPS sensor 110C, the geomagnetic sensor 111C, the atmospheric pressure sensor 112C, the acceleration sensor 113C, the angular velocity sensor 114C, the pulse sensor 115C, the temperature sensor 116C, the clocking section 160C, and the like, display processing (an example display control) for displaying an image on the display section 170C, sound output processing for outputting sounds from the sound output section 180C. The processing section 120C performs a process of receiving a control command from the electronic apparatus 1 via the communication section 190C, or various computation processes on data which is received from the electronic apparatus 1 via the communication section 190C, according to various programs. The processing section 120C performs a process of reading data from the storage section 130C, and transmitting the data to the electronic apparatus 1 in a predetermined format via the communication section 190C, according to various programs. The processing section 120C performs a process of transmitting various pieces of information to the electronic apparatus 1 via the communication section 190C, and displaying various screens on the basis of information received from the electronic apparatus 1, according to various programs. The processing section 120C performs other various control processes. For example, the processing section 120C performs a process of displaying images (images, moving images, text, symbols, and the like) on the display section 170C on the basis of at least some of the information received by the communication section 190C, the information received by the communication section 190C, and information stored in the storage section 130C. A vibration mechanism may be provided in the sensor device 1C, and various pieces of information may be converted into vibration information by the vibration mechanism so as to be presented to the user.

### 1-2-2. Configuration of Electronic Apparatus

As illustrated in FIG. 2, the electronic apparatus 1 is configured to include a GPS sensor 110, a geomagnetic sensor 111, an atmospheric pressure sensor 112, an acceleration sensor 113, an angular velocity sensor 114, a pulse sensor 115, a temperature sensor 116, a processing section 120, a storage section 130, an operation section 150, a clocking section 160, a display section 170, a sound output section 180, a communication section 190, and the like. However, the electronic apparatus 1 may have a configuration in which some of the constituent elements are deleted or changed, or may have a configuration in which other constituent elements (for example, a humidity sensor and an ultraviolet sensor) are added thereto.

The GPS sensor 110 is a sensor which generates positioning data (data such as latitude, longitude, altitude, and velocity vector) indicating a position and the like of the electronic apparatus 1 and outputs the data to the processing section 120, and is formed of, for example, a global positioning system (GPS) receiver. The GPS sensor 110 receives an electric wave including a satellite signal with a predetermined frequency bandwidth incoming from the outside by using a GPS antenna (not illustrated), extracts a GPS signal sent from a GPS satellite therefrom, and also generates positioning data indicating a position or the like of the electronic apparatus 1 on the basis of the GPS signal.

The geomagnetic sensor 111 is a sensor which detects a geomagnetic vector indicating a direction of a magnetic field of the earth, viewed from the electronic apparatus 1, and generates, for example, geomagnetic data indicating magnetic flux densities in three-axis directions which are orthogonal to each other. For example, a magnet resistive (MR) element, a magnet impedance (MI) element, or a hole element is used for the geomagnetic sensor 111.

The atmospheric pressure sensor 112 is a sensor which detects an ambient atmospheric pressure (atmospheric pressure), and includes, for example, a pressure sensitive element of a type (vibration type) using a change in a resonance frequency of a vibrator element. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a surface acoustic wave (SAW) vibrator. An output from the atmospheric pressure sensor 112 may be used to correct positioning data. The atmospheric pressure sensor 112 may be a MEMS type atmospheric pressure sensor manufactured by using, for example, a semiconductor manufacturing technique. Specifically, the atmospheric pressure sensor 112 includes a diaphragm portion which is bent and deformed by received pressure, and a distortion detection element which detects bending of the diaphragm portion. The diaphragm portion is made of, for example, silicon. The distortion detection element is, for example, a piezoelectric resistive element.

The acceleration sensor 113 is an inertial sensor which detects respective accelerations in the three-axis directions which intersect (are ideally orthogonal to) each other, and outputs digital signals (acceleration data) corresponding to magnitudes and directions of the detected three-axis accelerations. The outputs from the acceleration sensor 113 may be used to correct position information included in the positioning data from the GPS sensor 110.

The angular velocity sensor 114 is an inertial sensor which detects respective angular velocities in the three-axis directions which intersect (are ideally orthogonal to) each other, and outputs digital signals (angular velocity data) corresponding to magnitudes and directions of the measured three-axis angular velocities. The outputs from the angular velocity sensor 114 may be used to correct position information included in the positioning data from the GPS sensor 110.

The pulse sensor 115 is a sensor which generates a signal indicating a pulse of the user and outputs the signal to the processing section 120, and includes, for example, a light source such as a light emitting diode (LED) light source which applies measurement light having an appropriate wavelength toward a blood vessel under the skin, and a light receiving element which detects a change in the intensity of light generated at the blood vessel according to the measurement light.

The temperature sensor 116 is a deep body temperature sensor which detects a deep body temperature of a user's body. The temperature sensor 116 includes, for example, a base material, a first temperature sensor which measures the temperature of a first position in the base material as a first temperature, a second temperature sensor which measures the temperature of a second position which is different from the first position in the base material as a second temperature, a temperature sensor which measures the temperature of an outer shell body surrounding the base material as a medium temperature of the outer shell body inside, and a calculation portion which calculates a deep body temperature of a subject by using the first temperature, the second temperature, and the medium temperature. The temperature sensor 116 calculates a constant which correlates the medium temperature, the first temperature, and the second temperature with each other on the basis of the first temperature (Tb1) and the second temperature (Tp1) when the medium temperature is a first medium temperature (Tout1), the first temperature (Tb2) and the second temperature (Tp2) when the medium temperature is a second medium temperature (Tout2), and the first temperature (Tb3) and the second temperature (Tp3) when the medium temperature is a third medium temperature (Tout3), and calculates the deep body temperature (Tc). Each of such temperature sensors is, for example, a temperature sensitive element which outputs a signal (for example, a voltage corresponding to a temperature) corresponding to an ambient temperature. The temperature sensor may output a digital signal corresponding to a temperature. The temperature sensor has a pressure sensitive element of a type (vibration type) using a change in a resonance frequency of a vibrating element corresponding to an ambient temperature. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a SAW vibrator. Alternatively, the temperature sensor may be formed of a temperature sensitive element which detects a temperature with a thermocouple or a thermistor.

The storage section 130 is formed of, for example, various integrated circuit (IC) memories such as a read only memory (ROM), a flash ROM, and a random access memory (RAM), or a computer readable recording medium such as a hard disk or a memory card. The storage section 130 is formed of, for example, one or a plurality of IC memories, and includes a ROM storing data such as a program, and a RAM serving as a work region of the processing section 120. The RAM may include a nonvolatile RAM.

The operation section 150 is formed of, for example, a button, a key, a microphone, and a touch panel so as to have a voice recognition function (using the microphone (not illustrated)) and an action detection function (using the acceleration sensor 113 or the like), and performs a process of converting an instruction from the user into an appropriate signal which is then sent to the processing section 120.

The clocking section 160 is formed of, for example, a real time clock (RTC) IC or the like, and generates time data such as year, month, day, hour, minute, and second, and sends the data to the processing section 120.

The display section 170 is formed of, for example, a liquid crystal display (LCD), an organic electroluminescence (EL) display, an electrophoretic display (EPD), or a touch panel display, and displays various images in response to instructions from the processing section 120.

The sound output section 180 is formed of, for example, a speaker, a buzzer, or a vibrator, and generates various sounds (or vibration) in response to instructions from the processing section 120.

The communication section 190 performs a variety of control for establishing data communication between the electronic apparatus 1 and the sensor device 1C or the information terminal 2. The communication section 190 is configured to include a transceiver based on a short-range wireless communication standard such as Bluetooth (registered trademark) (including Bluetooth Low Energy (BTLE)), Wi-Fi (registered trademark) (Wireless Fidelity), Zigbee (registered trademark), near field communication (NFC), or ANT+(registered trademark).

The processing section 120 is formed of, for example, a micro processing unit (MPU), a digital signal processor (DSP), and an application specific integrated circuit (ASIC). The processing section 120 performs various processes according to a program stored in the storage section 130, and various commands which are input by the user via the operation section 150. The processes in the processing section 120 include, for example, data processing on data which is generated by the GPS sensor 110, the geomagnetic sensor 111, the atmospheric pressure sensor 112, the acceleration sensor 113, the angular velocity sensor 114, the pulse sensor 115, the temperature sensor 116, the clocking section 160, and the like, display processing for displaying an image on the display section 170, and sound output processing for outputting sounds from the sound output section 180. The processing section 120 performs a process of receiving a control command from the information terminal 2 via the communication section 190, or various computation processes on data which is received from the information terminal 2 via the communication section 190, according to various programs. The processing section 120 performs a process of reading data from the storage section 130, and transmitting the data to the information terminal 2 in a predetermined format via the communication section 190, according to various programs. The processing section 120 performs a process of transmitting various pieces of information to the information terminal 2 via the communication section 190, and displaying various screens on the basis of information received from the information terminal 2, according to various programs. The processing section 120 performs other various control processes. For example, the processing section 120 performs a process of displaying images (images, moving images, text, symbols, and the like) on the display section 170 on the basis of at least some of the information received by the communication section 190, the information received by the communication section 190, and information stored in the storage section 130. A vibration mechanism may be provided in the electronic apparatus 1, and various pieces of information may be converted into vibration information by the vibration mechanism so as to be presented to the user.

### 1-2-3. Configuration of Information Terminal

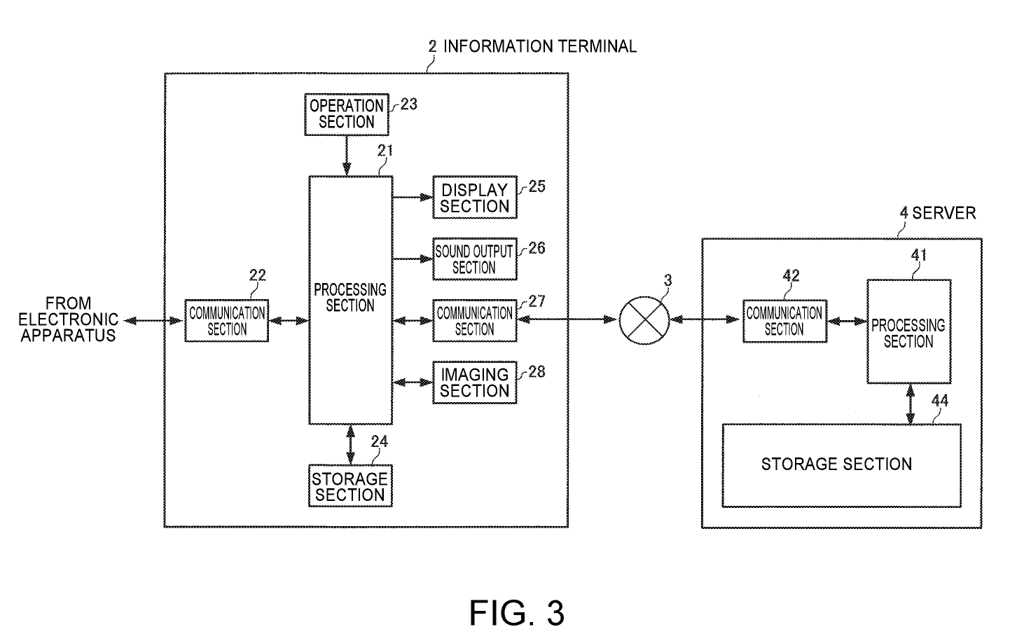

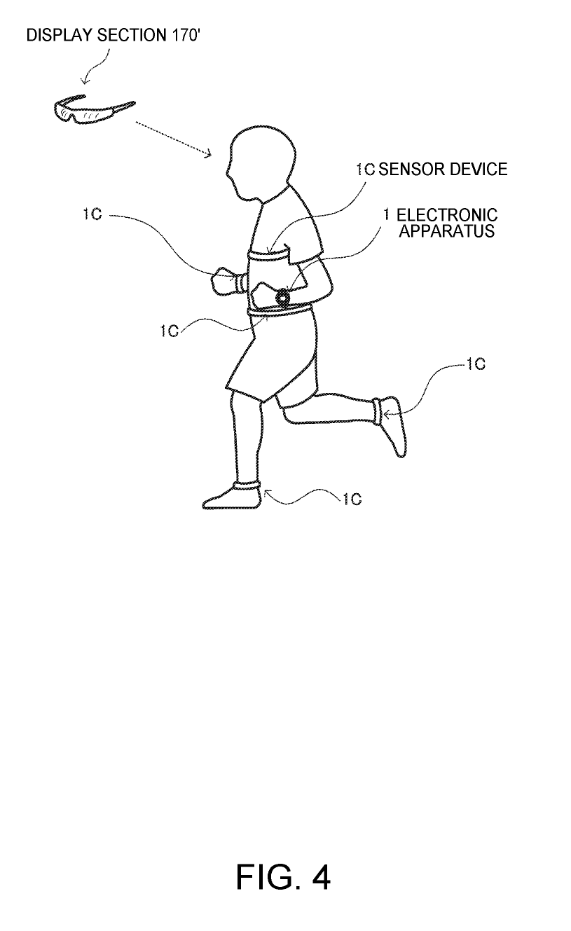

As illustrated in FIG. 3, the information terminal is configured to include a processing section 21, a communication section 22, an operation section 23, a storage section 24, a display section 25, a sound output section 26, a communication section 27, and an imaging section 28. However, the information terminal 2 may have a configuration in which some of the constituent elements are deleted or changed as appropriate, or may have a configuration in which other constituent elements are added thereto.

The communication section 22 performs a process of receiving data (measured data) or the like transmitted from the electronic apparatus 1 in a predetermined format and sending the data to the processing section 21, a process of transmitting a control command from the processing section 21 to the electronic apparatus 1 or the sensor device 1C, or the like.

The operation section 23 performs a process of acquiring data corresponding to the user's operation, and sending the data to the processing section 21. The operation section 23 may be, for example, a touch panel display, a button, a key, and a microphone.

The storage section 24 is formed of, for example, various integrated circuit (IC) memories such as a read only memory (ROM), a flash ROM, and a random access memory (RAM), or a computer readable recording medium such as a hard disk or a memory card. The storage section 24 stores programs for the processing section 21 performing various computation processes or control processes, various programs or data for realizing application functions. The storage section 24 is used as a work region of the processing section 21, and temporarily stores data which is input from the operation section 23, results of calculation executed by the processing section 21 according to various programs, and the like. The storage section 24 may store data which is required to be preserved for a long period of time among data items generated through processing in the processing section 21.

The display section 25 displays a processing result in the processing section 21 as text, a graph, a table, animation, and other images. The display section 25 may be, for example, a cathode ray tube (CRT), a liquid crystal display (LCD), a touch panel display, and a head mounted display (HMD). A single touch panel display may realize functions of the operation section 23 and the display section 25.

The sound output section 26 outputs a processing result in the processing section 21 as a sound (or vibration) such as a voice or a buzzer sound. The sound output section 26 may be, for example, a speaker or a buzzer.

The communication section 27 performs data communication with a communication section 42 of the server 4 via the network 3. For example, the communication section 27 performs a process of receiving data from the processing section 21 and transmitting the data to the communication section 42 of the server 4 in a predetermined format. For example, the communication section 27 performs a process of receiving information required to display a screen from the communication section 42 of the server 4 and sending the information to the processing section 21, or a process of receiving various pieces of information from the processing section 21 and transmitting the information to the communication section 42 of the server 4.

The imaging section 28 is a camera including a lens, a color imaging element, a focus adjustment mechanism, and the like, and generates a picture of a field generated by the lens as an image with the imaging element. Data (image data) regarding the image acquired by the imaging element is sent to the processing section 21 so as to be preserved in the storage section 24 or displayed on the display section 25.

The processing section 21 (an example of a computer) is formed of, for example, a central processing unit (CPU), a digital signal processor (DSP), and an application specific integrated circuit (ASIC). The processing section 21 performs various processes according to a program stored in the storage section 24, and various commands which are input by the user via the operation section 23. The processes in the processing section 21 include, for example, data processing on data which is generated by the electronic apparatus 1 or the sensor device 1C, display processing for displaying an image on the display section 25, sound output processing for outputting sounds from the sound output section 26, and image processing on an image acquired by the imaging section 28. The processing section 21 may be formed of a single processor, and may be formed of a plurality of processors. The processing section 21 performs a process of transmitting a control command to the electronic apparatus 1 via the communication section 22, or various computation processes on data which is received from the electronic apparatus 1 via the communication section 22, according to various programs. The processing section 21 performs a process of reading data from the storage section 24, and transmitting the data to the server 4 in a predetermined format via the communication section 27, according to various programs. The processing section 21 performs a process of transmitting various pieces of information to the server 4 via the communication section 27, and displaying various screens on the basis of information received from the server 4, according to various programs. The processing section 21 performs other various control processes. For example, the processing section 21 performs a process of displaying images (images, moving images, text, symbols, and the like) on the display section 25 on the basis of at least some of the information received by the communication section 27, the information received by the communication section 22, and information stored in the storage section 24. A vibration mechanism may be provided in the information terminal 2, the electronic apparatus 1, or the sensor device 1C, and various pieces of information may be converted into vibration information by the vibration mechanism so as to be presented to the user.

### 1-2-4. Configuration of Server

As illustrated in FIG. 3, the server 4 is configured to include a processing section 41, a communication section 42, and a storage section 44. However, the server 4 may have a configuration in which some of the constituent elements are deleted or changed as appropriate, or may have a configuration in which other constituent elements are added thereto.

The storage section 44 is formed of, for example, various integrated circuit (IC) memories such as a read only memory (ROM), a flash ROM, and a random access memory (RAM), or a computer readable recording medium such as a hard disk or a memory card. The storage section 44 stores a program for the processing section 41 performing various calculation processes or a control process, or various programs or data for realizing application functions. The storage section 44 is used as a work region of the processing section 41, and temporarily stores results of calculation executed by the processing section 41 according to various programs, and the like. The storage section 44 may store data which is required to be preserved for a long period of time among pieces of data generated through processing of the processing section 41. Various pieces of information stored in the storage section 44 will be described later.

The communication section 42 performs data communication with the communication section 27 of the information terminal 2 via the network 3. For example, the communication section 42 performs a process of receiving data from the communication section 27 of the information terminal 2, and sending the data to the processing section 41. For example, the communication section 42 performs a process of transmitting information required to display a screen to the communication section 27 of the information terminal 2 in a predetermined format, or a process of receiving information from the communication section 27 of the information terminal 2 and sending the information to the processing section 41.

The processing section 41 performs a process of receiving data from the information terminal 2 via the communication section 42 and storing the data in the storage section 44, according to various programs. The processing section 41 performs a process of receiving various pieces of information from the information terminal 2 via the communication section 42, and transmitting information required to display various screens to the information terminal 2, according to various programs. The processing section 41 performs other various control processes.

### 1-3. Method of Living Body Monitoring Method

The system of the present embodiment includes a measurement unit which measures a deep body temperature of a user's body, a comparison unit which compares the deep body temperature with a predetermined comparison reference, a generation unit which generates information regarding a physical condition on the basis of a result of the comparison, and a display which presents the information to the user.

In the system of the present embodiment, elements which can operate as the measurement unit are the temperature sensor 116C of the sensor device 1C, the temperature sensor 116 of the electronic apparatus 1, and the like. Elements which can operate as the comparison unit and the generation unit are the processing section 120C of the sensor device 1C, the processing section 120 of the electronic apparatus 1, the processing section 21 of the information terminal 2, and the like. Elements which can operate as the display are the display section 170C and the sound output section 180C of the sensor device 1C, the display section 170 and the sound output section 180 of the electronic apparatus 1, the display section 25 and the sound output section 26 of the information terminal 2, and the like.

Here, the information presented to the user by the display is information based on a result of comparison with a predetermined comparison reference, and can thus be more effectively used for the user's life or training than information indicating a deep body temperature itself. This is because an action to be performed by a user completely differs depending on a range in which the deep body temperature is included. Therefore, for example, the user may use the information as a reference when determining whether or not the content of the user's life is reconsidered or whether or not the content of the user's training is reconsidered.

Hereinafter, the system of the present embodiment is assumed to have a mode (daily life mode) in which measurement regarding a user's life is performed, and a mode (training mode) in which measurement regarding the user's training is performed.

### 1-3-1. Mounting Example on User

For example, in a case where a user wishes to intensively train a specific part (for example, the abdomen) of the body, the sensor device 1C may be mounted on the part (abdomen). The sensor device 1C has the temperature sensor 116C, and can thus measure a deep body temperature (muscle temperature) of the part (abdomen).

However, since it is hard for the user to operate or view the sensor device 1C mounted on the abdomen during training, in this case, the user may mount the electronic apparatus 1 on the wrist, and may remotely operate the sensor device 1C via the electronic apparatus 1 by using the electronic apparatus 1 as a master device. In this case, for example, the sensor device 1C periodically performs communication with the electronic apparatus 1 as a master device, and, for example, measured data (for example, at least an output from the temperature sensor 116C) acquired by the sensor device 1C is sequentially transmitted to the electronic apparatus 1.

The user may separately mount a plurality of sensor devices 1C on a plurality of parts of the body of the user.

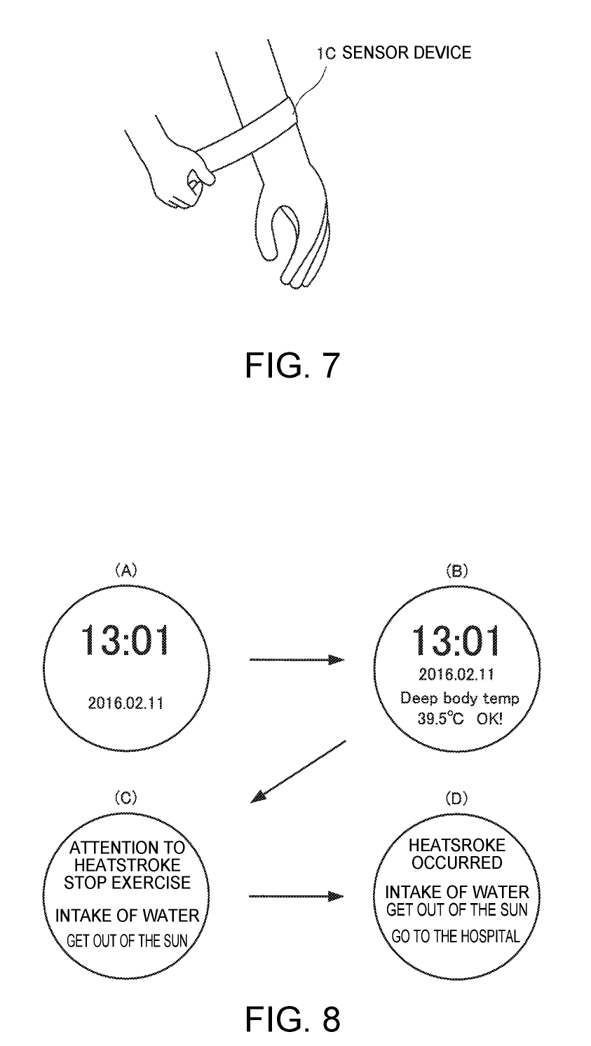

The sensor device 1C may have various forms such as a medal form, a belt form, and a seal form. The sensor device 1C illustrated in FIG. 6 has a belt form, and the sensor device 1C illustrated in FIG. 7 has a seal form. In the present embodiment, a sensor to be disposed at a position close to the user's body is mainly a temperature sensor, and thus entire sensor device 1C may not have a seal form or a belt form but only the temperature sensor 116C may have a seal form or a belt form. FIG. 7 illustrates the thin sensor device 1C, but, in a case where a location where a sensor unit as a part of the sensor device 1C is disposed is required to be separated from the user's body to some extent or more, the sensor device 1C may be made thick, and then the sensor unit may be provided in a portion separated from the body in the sensor device 1C.

In the present system, in a case where there is a manager observing the life of a user or a coach coaching training of the user, a location to which information is presented by the display may be set to the information terminal 2, and the information terminal 2 may be operated by the manager or the coach. In this case, at least the measurement unit may be mounted on the sensor device 1C or the electronic apparatus 1, and at least the display may be mounted on the information terminal 2. The information terminal 2 is an example of an information terminal which can be operated by other users.

In a case where there is no particular part desired to be intensively trained even if the present system is operated in the training mode, or the present system is operated in the daily life mode, the user may wear, for example, only the electronic apparatus 1 without using the sensor device 1C. In this case, the electronic apparatus 1 includes a measurement unit which measures a deep body temperature of a user's body, a comparison unit which compares the deep body temperature with a predetermined comparison reference, a generation unit which generates information regarding a physical condition on the basis of a result of the comparison, and a display which presents the information to the user. In this case, the temperature sensor 116 of the electronic apparatus 1 operates as the measurement unit, the processing section 120 of the electronic apparatus 1 operates as the comparison unit and the generation unit, and the display section 170 and the sound output section 180 of the electronic apparatus 1 operate as the display.

Hereinafter, a description will be made of a case where all functions of the measurement unit, the comparison unit, the generation unit, and the display are installed in the electronic apparatus 1. In this case, the temperature sensor 116 of the electronic apparatus 1 operates as the measurement unit, the processing section 120 of the electronic apparatus 1 operates as the comparison unit and the generation unit, and the display section 170 and the sound output section 180 of the electronic apparatus 1 operate as the display.

Hereinafter, the user operates the operation section 150 of the electronic apparatus 1 as necessary, and can thus change a mode of the electronic apparatus 1 between the daily life mode and the training mode.

### 1-3-2. Display Examples on HMD

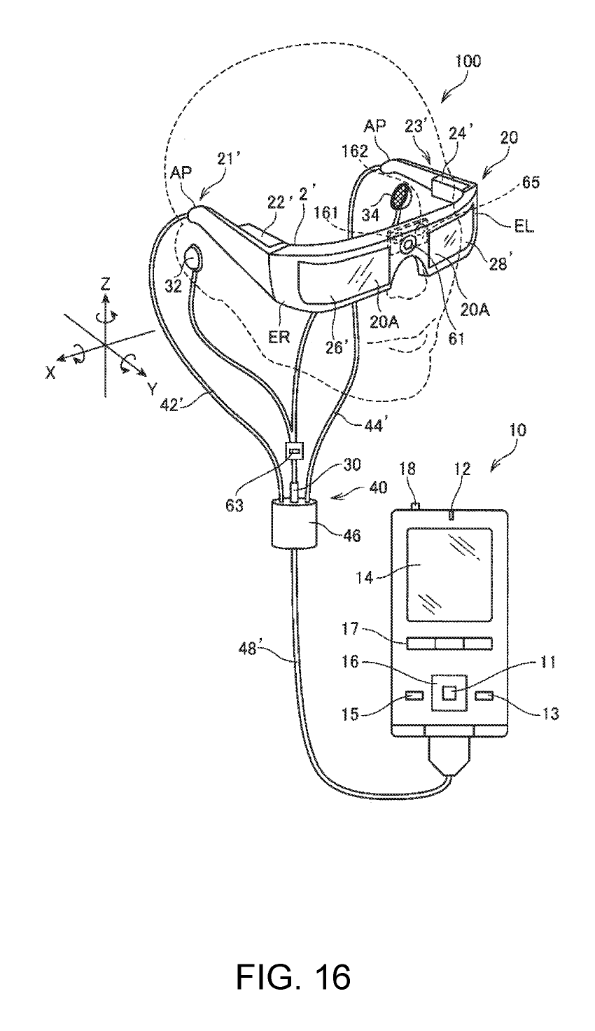

The user may use a head mounted type display section 170′ called a head mounted display (HMD) which is provided separately from the electronic apparatus 1, instead of the display section 170 of the electronic apparatus 1. The head mounted type display section 170′ performs data communication with the electronic apparatus 1, receives image display data from the electronic apparatus 1, and displays an image in front of the eyes of the user on the basis of the data. As the display section 170′, for example, a head mounted display (refer to FIG. 16) which will be described later may be used.

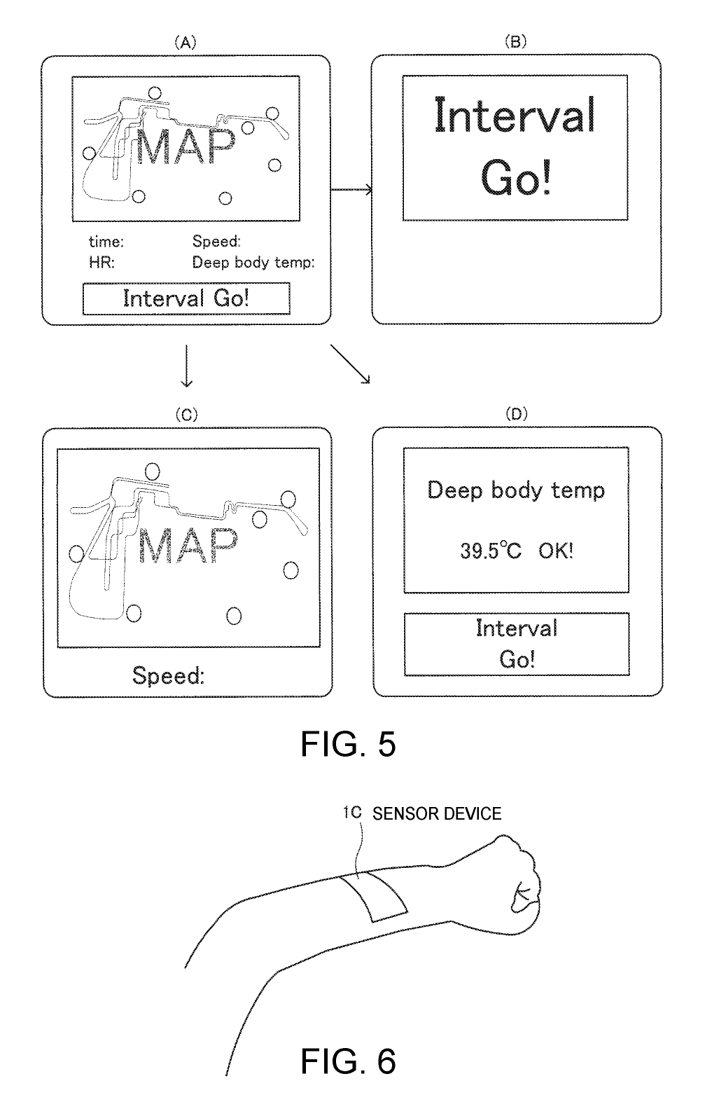

A shape of a display screen (a virtual image which will be described later) displayed in front of the eyes of the user, by the head mounted type display section 170′, has a substantially rectangular shape, for example, as illustrated in FIG. 5. The examples illustrated in FIG. 5 show states in which a text image for a notification, a map image, and the like are displayed on the display section 170′. The examples illustrated in FIG. 5 show states in which a map image is displayed on a display screen as necessary. The map (in each figure, a map image is illustrated by a rectangular frame with text “MAP” or “Map”; this is also the same for the following description) is a map of a region including a point where the user is present. The map is used in a case where the user checks the present point, or the electronic apparatus 1 guides (navigates) the user to a destination.

FIG. 5(A) illustrates an example in which two or more pieces of measured data (a time point (time), a speed (speed), a heart rate (HR), and a deep body temperature (Deep body temperature)), a map, and predetermined information (“Interval Go!”) to be presented to the user are displayed; FIG. 5(B) illustrates an example in which only the predetermined information (“Interval Go!”) to be presented to the user is displayed; FIG. 5(C) illustrates an example in which the image and a text image of one (speed) of the pieces of measured data are displayed; and FIG. 5(D) illustrates an example in which text image of one (deep body temperature) of the pieces of measured data and the predetermined information (“Interval Go!”) to be presented to the user are displayed. Information to be displayed on a screen, a layout of information, or the like may be customized by the user, and may be automatically determined depending on a size of a screen, or the like.

A screen displayed on the head mounted type display section 170′ and a screen displayed on the display section 170 built into the electronic apparatus 1 may be different from each other in terms of an amount of information which can be collectively displayed, a screen size, a layout of information, or the like, but the content of information to be displayed is fundamentally the same. Hereinafter, a case is assumed in which the display section 170 built into the electronic apparatus 1 is used. In FIG. 8 or the like which will be described later, a contour shape of the built-in type display section 170 is a circular shape (refer to FIG. 8 or the like).

### 1-3-3. Daily Life Mode

### 1-3-3-1. Clock Display Screen

The user sets the electronic apparatus 1 to the daily life mode during non-training.

In the processing section 120 of the electronic apparatus 1 in the daily life mode drives, for example, the GPS sensor 110, the geomagnetic sensor 111, the atmospheric pressure sensor 112, the acceleration sensor 113, the angular velocity sensor 114, the pulse sensor 115, and the temperature sensor 116, for example, at a predetermined frequency, and writes measured data acquired by the sensors into the storage section 130 in correlation with acquisition time points. The measured data accumulated in the storage section 130 can be passed to the information terminal 2 as necessary, and may also be uploaded to the server 4. The uploaded measured data is stored in the storage section 44 of the server 4 for each user.

The processing section 120 of the electronic apparatus 1 in the daily life mode fundamentally displays a clock display screen as illustrated in, for example, FIG. 8(A) on the display section 170 in order to reduce power consumption. This is because, during display of the clock display screen, driving of at least one of the GPS sensor 110, the geomagnetic sensor 111, the atmospheric pressure sensor 112, the acceleration sensor 113, the angular velocity sensor 114, the pulse sensor 115, and the temperature sensor 116 can be paused.

The electronic apparatus 1 in the daily life mode compares measured data (deep body temperature) acquired by at least the temperature sensor 116 with a threshold value for attracting attention and a threshold value for warning, and, for example, periodically determines whether or not the deep body temperature exceeds the threshold value for attracting attention and whether or not the deep body temperature exceeds the threshold value for warning. The threshold value for attracting attention and the threshold value for warning will be described later.

### 1-3-3-2. Measured Data Display Screen

In a case where a predetermined instruction is received from the user via the operation section 150, the processing section 120 of the electronic apparatus 1 changes a display screen on the display section 170 to a measured data display screen as illustrated in FIG. 8(B). A numerical value of measured data acquired by any one of the sensors mounted on the electronic apparatus 1 is displayed on the measured data display screen. FIG. 8(B) illustrates an example in which measured data (deep body temperature) acquired by the temperature sensor 116 is displayed.

The measured data display screen may include anyone of measured data (position) acquired by the GPS sensor 110, measured data (azimuth) acquired by the geomagnetic sensor 111, measured data (atmospheric pressure) acquired by the atmospheric pressure sensor 112, and measured data (heartbeat) acquired by the pulse sensor 115. The processing section 120 of the electronic apparatus 1 may allow the user to select in advance one or a plurality of pieces of measured data to be displayed on the measured data display screen (display selection mode).

### 1-3-3-3. Alert Screen

The processing section 120 of the electronic apparatus 1 displays an alert screen as illustrated in FIG. 8(C) on the display section 170 in a case where the deep body temperature of the user exceeds the threshold value for attracting attention. The alert screen is an example of information regarding a physical condition of the user.

The alert screen includes advice to the user, and the advice includes at least one of advice related to the necessity of water intake, advice related to the necessity of rest, advice related to the necessity of atmospheric temperature adjustment, advice related to the necessity of body temperature adjustment, and advice related to the necessity of doctor's examination.

The alert screen illustrated in FIG. 8(C) includes alert, “attention to heatstroke”, the advice related to the necessity of water intake, “intake of water”, the advice related to the necessity of rest, “stop exercise”, and the advice related to the necessity of atmospheric temperature adjustment, “get out of the sun”.

The processing section 120 of the electronic apparatus 1 may output at least one of a voice for attracting attention, light for attracting attention, vibration for attracting attention, and motion for attracting attention, in addition to the display of the alert screen. The light is output via the display section 170, and the voice, the vibration, and the motion are output via the sound output section 180.

In other words, in the present system, information presented to the user may be information which can be recognized by the user through at least one of an auditory sense, a visual sense, and a tactile sense. In this case, the information can be recognized by the user through at least one of the user's auditory sense, visual sense, and tactile sense. In the system of the present embodiment, information presented to the user may be displayed by using at least one of a color, text, a symbol, a scale, and a pattern which can be recognized through a visual sense by the user. In this case, the information can be recognized by the user by using at least one of a color, text, a symbol, a scale, and a pattern.

For example, the processing section 120 may combine a scale image with a pointer image so as to display a deep body temperature (to display a gauge or a meter), provide an alert zone from the threshold value for attracting attention to the threshold value for warning in the scale image, and highlight a color or a pattern of the alert zone. In a case where the pointer image indicating the deep body temperature is located in the alert zone, a notification using light or a sound may be performed.

The processing section 120 of the electronic apparatus 1 may allow the user to designate whether or not a voice, light, vibration, or motion for attracting attention is output.

The processing section 120 of the electronic apparatus 1 may allow the user to designate the threshold value for attracting attention. A designatable range is set to a range of, for example, 37° C. to 40° C.

The user's instruction or designation is input to the electronic apparatus 1 via the operation section 150.

### 1-3-3-4. Warning Screen

The processing section 120 of the electronic apparatus 1 changes a screen to a warning screen as illustrated in FIG. 8(D) in a case where the deep body temperature of the user exceeds the threshold value for warning. The warning screen is also an example of information regarding a physical condition of the user. The threshold value for warning is assumed to be greater than the threshold value for attracting attention.

The warning screen also includes advice to the user, and the advice includes at least one of advice related to the necessity of water intake, advice related to the necessity of rest, advice related to the necessity of atmospheric temperature adjustment, advice related to the necessity of body temperature adjustment, and advice related to the necessity of doctor's examination. However, the warning screen includes firmer advice (advice closer to “having to do” than “being better to do”) than in the alert screen.

The warning screen illustrated in FIG. 8(D) includes a warning, “You had heatstroke”, the advice related to the necessity of water intake, “intake of water”, the advice related to the necessity of atmospheric temperature adjustment, “get out of the sun”, and the advice related to the necessity of doctor's examination, “go to the hospital”.

Therefore, it is possible to recommend, to the user, any one of water intake, rest, atmospheric temperature adjustment, body temperature adjustment, and doctor's examination at right timing corresponding to a physical condition of the user. According to the advice, even in a case where the user's deep body temperature is too high, the user's bodily functions are reduced, and thus the user's judgment ability deteriorates, it is possible to prompt the user to perform a right action.

The processing section 120 of the electronic apparatus 1 may output at least one of a voice for warning, light for warning, vibration for warning, and motion for warning, in addition to the display of the warning screen. The light is output via the display section 170, and the voice, the vibration, and the motion are output via the sound output section 180.

In other words, in the present system, information presented to the user may be information which can be recognized by the user through at least one of an auditory sense, a visual sense, and a tactile sense. In this case, the information can be recognized by the user through at least one of the user's auditory sense, visual sense, and tactile sense. In the system of the present embodiment, information presented to the user may be displayed by using at least one of a color, text, a symbol, a scale, and a pattern which can be recognized through a visual sense by the user. In this case, the information can be recognized by the user by using at least one of a color, text, a symbol, a scale, and a pattern.

For example, the processing section 120 may combine a scale image with a pointer image so as to display a deep body temperature (to display a gauge or a meter), provide an alert zone in the scale image, and perform a notification using light or a sound when the pointer image indicating the deep body temperature exceeds an upper limit of the alert zone. This notification is preferably highlighted more than a notification performed when the pointer image is included in the alert zone.

The processing section 120 of the electronic apparatus 1 may allow the user to designate whether or not a voice, light, vibration, or motion for warning is output.

The processing section 120 of the electronic apparatus 1 may allow the user to designate the threshold value for warning. A designatable range is set to a range of, for example, 40° C. to 42° C.

The user's instruction or designation is input to the electronic apparatus 1 via the operation section 150.

### 1-3-4. Training Mode

### 1-3-4-1. Measured Data Display Screen

The user sets the electronic apparatus 1 to the training mode during training. Herein, as an example of the training mode, a mode for an interval run is assumed, and a description will be made of a case where the electronic apparatus 1 notifies the user of a timing at which high-speed running (main training) of the interval run is started, a timing at which the high-speed running (main training) is stopped, and a timing at which the high-speed running (main training) is resumed.

Even though the user performs warm-up before starting the interval run, if a muscle temperature (equivalent to a deep body temperature) does not reach a temperature at which the maximum oxygen intake occurs due to an outdoor air temperature, as a result, training is performed on a low load so that a specific stimulus cannot be applied to the body, and therefore physical ability improvement (training effect) based on subsequent overcompensation cannot be expected. A heat stroke may occur due to an increase in a deep body temperature depending on the training content or an environment in which training is performed.

First, the electronic apparatus 1 in the training mode drives, for example, the GPS sensor 110, the geomagnetic sensor 111, the atmospheric pressure sensor 112, the acceleration sensor 113, the angular velocity sensor 114, the pulse sensor 115, and the temperature sensor 116, for example, at a predetermined frequency, and writes measured data acquired by the sensors into the storage section 130 in correlation with acquisition time points. The measured data accumulated in the storage section 130 can be passed to the information terminal 2 as necessary, and may also be uploaded to the server 4. The uploaded measured data is stored in the storage section 44 of the server 4 for each user.

The electronic apparatus 1 in the training mode compares at least measured data (deep body temperature) acquired by the temperature sensor 116 with a threshold value for starting an exercise and a threshold value for stopping an exercise, and, for example, periodically determines whether or not a deep body temperature exceeds the threshold value for starting an exercise, whether or not the deep body temperature exceeds the threshold value for stopping an exercise, and whether or not the deep body temperature is reduced to a threshold value for resuming an exercise. The threshold value for starting an exercise, the threshold value for stopping an exercise, and the threshold value for resuming an exercise will be described later.

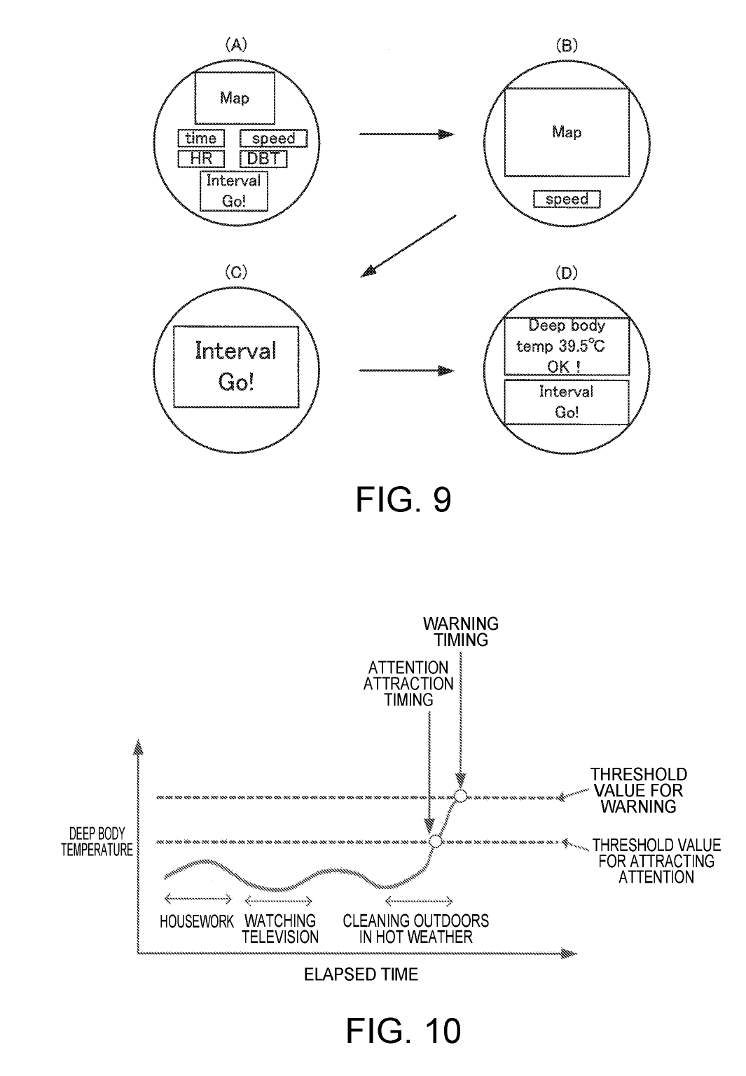

The processing section 120 of the electronic apparatus 1 in the training mode fundamentally displays a measured data display screen as illustrated in FIG. 9(A) or 9(B) on the display section 170.

The measured data display screen displays, as illustrated in FIG. 9(A) or 9(B), a map (Map) of a region where the user is present, a time point (time), a moving speed (speed), a heart rate (HR), a deep body temperature (DBT), and information indicating whether or not an exercise is possible (“Interval Go!”).

The measured data display screen may include any one of measured data (position) acquired by the GPS sensor 110, measured data (azimuth) acquired by the geomagnetic sensor 111, measured data (atmospheric pressure) acquired by the atmospheric pressure sensor 112, and measured data (heartbeat) acquired by the pulse sensor 115. The processing section 120 of the electronic apparatus 1 may allow the user to select in advance one or a plurality of pieces of measured data to be displayed on the measured data display screen (display selection mode).

### 1-3-4-2. Exercise Starting Screen

The processing section 120 of the electronic apparatus 1 displays an exercise starting screen as illustrated in FIG. 9(C) or 9(D) on the display section 170 in a case where a deep body temperature of the user exceeds the threshold value for starting an exercise. The exercise starting screen is an example of information regarding a physical condition of the user.

The exercise starting screen (FIG. 9(C) or 9(D)) includes advice to the user, and the advice includes advice related to whether or not starting of an exercise is possible, specifically, advice on exercise starting, “Interval Go!”.

Therefore, it is possible to prompt the user to start an exercise at a right timing corresponding to a physical condition of the user.

The processing section 120 of the electronic apparatus 1 may output at least one of a voice for starting an exercise, light for starting an exercise, vibration for starting an exercise, and motion for starting an exercise, in addition to the display of the exercise starting screen. The light is output via the display section 170, and the voice, the vibration, and the motion are output via the sound output section 180.

In other words, in the present system, information presented to the user may be information which can be recognized by the user through at least one of an auditory sense, a visual sense, and a tactile sense. In this case, the information can be recognized by the user through at least one of the user's auditory sense, visual sense, and tactile sense. In the system of the present embodiment, information presented to the user may be displayed by using at least one of a color, text, a symbol, a scale, and a pattern which can be recognized through a visual sense by the user. In this case, the information can be recognized by the user by using at least one of a color, text, a symbol, a scale, and a pattern.

For example, the processing section 120 may combine a scale image with a pointer image so as to display a deep body temperature (to display a gauge or a meter), provide an exercise zone with the threshold value for starting an exercise as a lower limit and the threshold value for stopping an exercise as an upper limit in the scale image, and highlight a color or a pattern of the exercise zone. A notification using light or a sound may be performed at a timing at which the pointer image indicating the deep body temperature exceeds the lower limit of the exercise zone.

The processing section 120 of the electronic apparatus 1 may allow the user to designate whether or not a voice, light, vibration, or motion for starting an exercise is output.

The processing section 120 of the electronic apparatus 1 may allow the user to designate the threshold value for starting an exercise. A designatable range is set to a range of, for example, 37° C. to 40° C.

The user's instruction or designation is input to the electronic apparatus 1 via the operation section 150.

### 1-3-4-3. Exercise Stopping Screen

The processing section 120 of the electronic apparatus 1 displays an exercise stopping screen (not illustrated) on the display section 170 in a case where a deep body temperature of the user exceeds the threshold value for stopping an exercise after the exercise starting screen is displayed (that is, after the main training is started).

The exercise stopping screen includes advice to the user, and the advice includes advice related to whether or not an exercise is required to be stopped, specifically, advice on exercise stoppage, “Interval Stop!”.

Therefore, it is possible to prompt the user to stop an exercise at a right timing corresponding to a physical condition of the user.

The processing section 120 of the electronic apparatus 1 may output at least one of a voice for stopping an exercise, light for stopping an exercise, vibration for stopping an exercise, and motion for stopping an exercise, in addition to the display of the exercise stopping screen. The light is output via the display section 170, and the voice, the vibration, and the motion are output via the sound output section 180.

In other words, in the present system, information presented to the user may be information which can be recognized by the user through at least one of an auditory sense, a visual sense, and a tactile sense. In this case, the information can be recognized by the user through at least one of the user's auditory sense, visual sense, and tactile sense. In the system of the present embodiment, information presented to the user may be displayed by using at least one of a color, text, a symbol, a scale, and a pattern which can be recognized through a visual sense by the user. In this case, the information can be recognized by the user by using at least one of a color, text, a symbol, a scale, and a pattern.

For example, the processing section 120 may combine a scale image with a pointer image so as to display a deep body temperature (to display a gauge or a meter), provide an exercise zone in the scale image, and perform a notification using light or a sound at a timing at which the pointer image exceeds an upper limit of the exercise zone.

The processing section 120 of the electronic apparatus 1 may allow the user to designate whether or not a voice, light, vibration, or motion for stopping an exercise is output.

The processing section 120 of the electronic apparatus 1 may allow the user to designate the threshold value for stopping an exercise. A designatable range is set to a range of, for example, 38° C. to 42° C.

The user's instruction or designation is input to the electronic apparatus 1 via the operation section 150.

### 1-3-4-4. Exercise Resuming Screen

The processing section 120 of the electronic apparatus 1 displays an exercise resuming screen (not illustrated) on the display section 170 in a case where a deep body temperature of the user is reduced to the threshold value for resuming an exercise after the exercise stopping screen is displayed (that is, after the main training is stopped). The threshold value for resuming an exercise is set to be the same as, for example, the threshold value for starting an exercise.

The exercise resuming screen includes advice to the user, and the advice includes advice related to whether or not an exercise is required to be resumed, specifically, advice on exercise resuming, “Interval Go!”.

Therefore, it is possible to prompt the user to resume an exercise at a right timing corresponding to a physical condition of the user.

The processing section 120 of the electronic apparatus 1 may output at least one of a voice for resuming an exercise, light for resuming an exercise, vibration for resuming an exercise, and motion for resuming an exercise, in addition to the display of the exercise resuming screen. The light is output via the display section 170, and the voice, the vibration, and the motion are output via the sound output section 180.

In other words, in the present system, information presented to the user may be information which can be recognized by the user through at least one of an auditory sense, a visual sense, and a tactile sense. In this case, the information can be recognized by the user through at least one of the user's auditory sense, visual sense, and tactile sense. In the system of the present embodiment, information presented to the user may be displayed by using at least one of a color, text, a symbol, a scale, and a pattern which can be recognized through a visual sense by the user. In this case, the information can be recognized by the user by using at least one of a color, text, a symbol, a scale, and a pattern.

For example, the processing section 120 may combine a scale image with a pointer image so as to display a deep body temperature (to display a gauge or a meter), provide an exercise zone in the scale image, and perform a notification using light or a sound at a timing at which the pointer image is reduced to a lower limit of the exercise zone.

The processing section 120 of the electronic apparatus 1 may allow the user to designate whether or not a voice, light, vibration, or motion for resuming an exercise is output.

The processing section 120 of the electronic apparatus 1 may allow the user to designate the threshold value for resuming an exercise. A designatable range is set to a range of, for example, 37° C. to 40° C.

The user's instruction or designation is input to the electronic apparatus 1 via the operation section 150.

### 1-3-5. Map Display

In the system of the present embodiment, information presented to the user may include at least one of information regarding a point where water intake is possible, information regarding a point where a rest is possible, information regarding a point where atmospheric temperature adjustment is possible, information regarding a point where body temperature adjustment is possible, and information regarding a point where a doctor's examination is possible. In this case, it is possible to notify the user of a point where the user takes any one of water intake, a rest, atmospheric temperature adjustment, body temperature adjustment, and a doctor's examination, at a right timing corresponding to a physical condition of the user.

For example, the processing section 120 of the electronic apparatus 1 may display a map (a map of a region including a point where the user is present) on the alert screen, the warning screen, or the exercise stopping screen, and may add a mark to a location corresponding to at least one of a point where water intake is possible, a point where a rest is possible, a point where atmospheric temperature adjustment is possible, a point where body temperature adjustment is possible, and a point where a doctor's examination is possible.

The processing section 120 of the electronic apparatus 1 may recognize a point where the user is present on the basis of measured data (positioning data) acquired by the GPS sensor 110. The processing section 120 of the electronic apparatus 1 may download map data in advance to the storage section 130 of the electronic apparatus 1 via the server 4 and the information terminal 2. The storage section 44 of the server 4 stores in advance data regarding a wide map on which information regarding a point where water intake is possible, a point where a rest is possible, a point where atmospheric temperature adjustment is possible, a point where body temperature adjustment is possible, and a point where a doctor's examination is possible is posted, and may transmit a part (data of a necessary region) of the data regarding the map to the information terminal 2 in response to a request from the information terminal 2.

The processing section 120 of the electronic apparatus 1 may guide the user toward any one (for example, a point closest to the present point of the user) of marked points by using a well-known navigation function. Navigation may be realized by using an image, sounds, or light, or a combination thereof.

### 1-3-6. Change in Deep Body Temperature in Daily Life Mode

FIG. 10 illustrates an example of a group indicating a temporal change in a deep body temperature in the daily life mode. Information, “housework”, “watching television”, and “cleaning outdoors in hot weather” illustrated on a lower part in FIG. 10 indicates actions of the user, and a line with arrowheads at both ends illustrated on the lower part in FIG. 10 indicates a substantial period in which the action is performed.

As illustrated in FIG. 10, when the electronic apparatus 1 is in the daily life mode, the user does housework or watches television. In the normal life such as housework or watching television, a deep body temperature of the user does not exceed the threshold value for attracting attention, but there is concern that the deep body temperature may considerably increase in a case where the user performs cleaning outdoors in hot weather.

However, in a case where a deep body temperature exceeds the threshold value for attracting attention, the electronic apparatus 1 of the present embodiment attracts user's attention by using an alert screen or the like. At this time, in a case where the user disregards the alert, and continues to perform cleaning outdoors, there is concern that the deep body temperature may further increase.

Thereafter, in a case where the deep body temperature exceeds the threshold value for warning, the electronic apparatus 1 of the present embodiment issues a warning to the user by using a warning screen or the like.

In other words, the electronic apparatus 1 in the daily life mode performs alert in a case where the deep body temperature of the user is included in the alert zone between the threshold value for warning and the threshold value for attracting attention, performs a warning in a case where the deep body temperature is included in a range higher than the alert zone, and does not perform a warning and alert in a case where the deep body temperature is included in a range lower than the alert zone.

### 1-3-7. Change in Deep Body Temperature in Training Mode

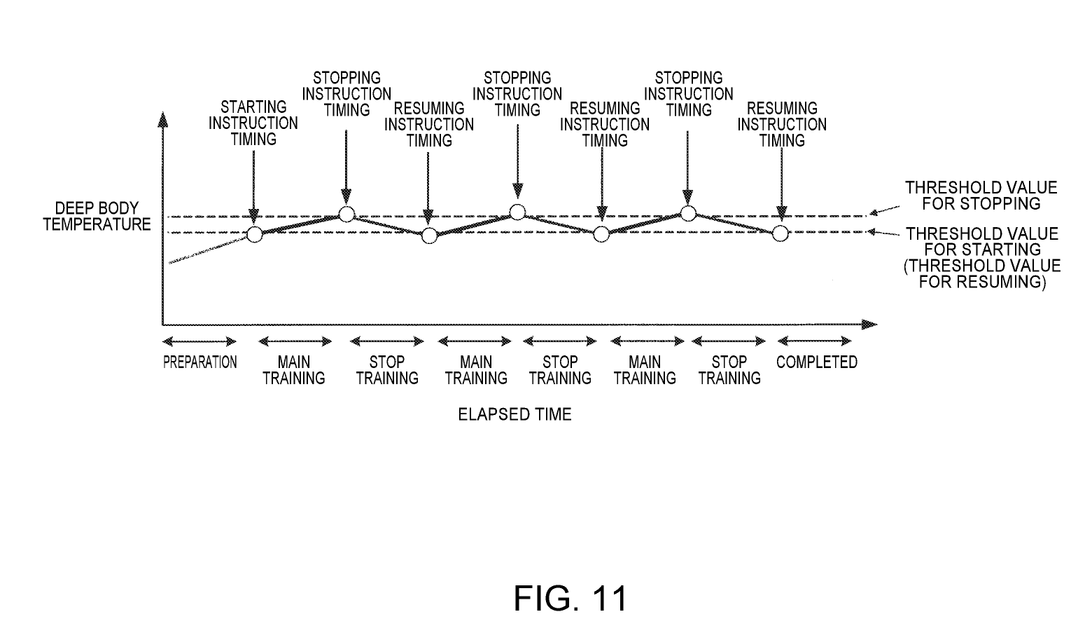

FIG. 11 is an example of a graph illustrating a temporal change in a deep body temperature in the training mode. Information, “preparation”, “main training”, “stop training”, and “completed” illustrated on a lower part in FIG. 11 indicates actions of the user, and a line with arrowheads at both ends illustrated on the lower part in FIG. 11 indicates a substantial period in which the action is performed.

As illustrated in FIG. 11, when the electronic apparatus 1 is in the training mode, the user performs an interval run of repeating main training and training stoppage. Before starting training, the user warms the body by performing a preparatory exercise such as stretching and jogging. In the present embodiment, the user determines a timing at which training is started, a timing at which the training is stopped, and a timing at which the training is resumed, according to advice of the electronic apparatus 1.

A deep body temperature of the user has a low value before a preparatory exercise, but the deep body temperature of the user gradually increases through the preparatory exercise. In a case where the deep body temperature exceeds the threshold value for starting an exercise, the electronic apparatus 1 gives an instruction for starting an exercise to the user by using an exercise starting screen or the like.

If the user starts main training, the deep body temperature of the user further increases. In a case where the deep body temperature exceeds the threshold value for stopping an exercise, the electronic apparatus 1 gives an instruction for stopping an exercise to the user by using an exercise stopping screen or the like.

If the user stops the training, the deep body temperature of the user is gradually reduced. In a case where the deep body temperature is reduced to the threshold value for resuming an exercise, the electronic apparatus 1 gives an instruction for resuming an exercise to the user by using an exercise resuming screen or the like.

If the user resumes the main training, the deep body temperature of the user further increases. In a case where the deep body temperature exceeds the threshold value for stopping an exercise, the electronic apparatus 1 gives an instruction for stopping an exercise to the user by using an exercise stopping screen or the like.

If the user stops the training, the deep body temperature of the user is gradually reduced. In a case where the deep body temperature is reduced to the threshold value for resuming an exercise, the electronic apparatus 1 gives an instruction for resuming an exercise to the user by using an exercise resuming screen or the like.

In other words, the electronic apparatus 1 in the training mode gives an instruction for starting an exercise in a case where the deep body temperature of the user reaches a lower limit of the exercise zone between the threshold value for starting an exercise and the threshold value for stopping an exercise before main training is started.

The electronic apparatus 1 in the training mode gives an instruction for stopping an exercise in a case where the deep body temperature of the user reaches an upper limit of the exercise zone between the threshold value for starting an exercise and the threshold value for stopping an exercise after starting main training.

The electronic apparatus 1 in the training mode gives an instruction for resuming an exercise in a case where the deep body temperature of the user is reduced to a lower limit of the exercise zone between the threshold value for stopping an exercise and the threshold value for resuming an exercise (=the threshold value for starting an exercise) after the main training is stopped.

Therefore, the electronic apparatus 1 in the training mode gives an instruction for starting, stopping, and resuming main training so that a state in which the deep body temperature of the user is included in the exercise zone is maintained.

### 1-3-8. Flow of Process in Daily Life Mode

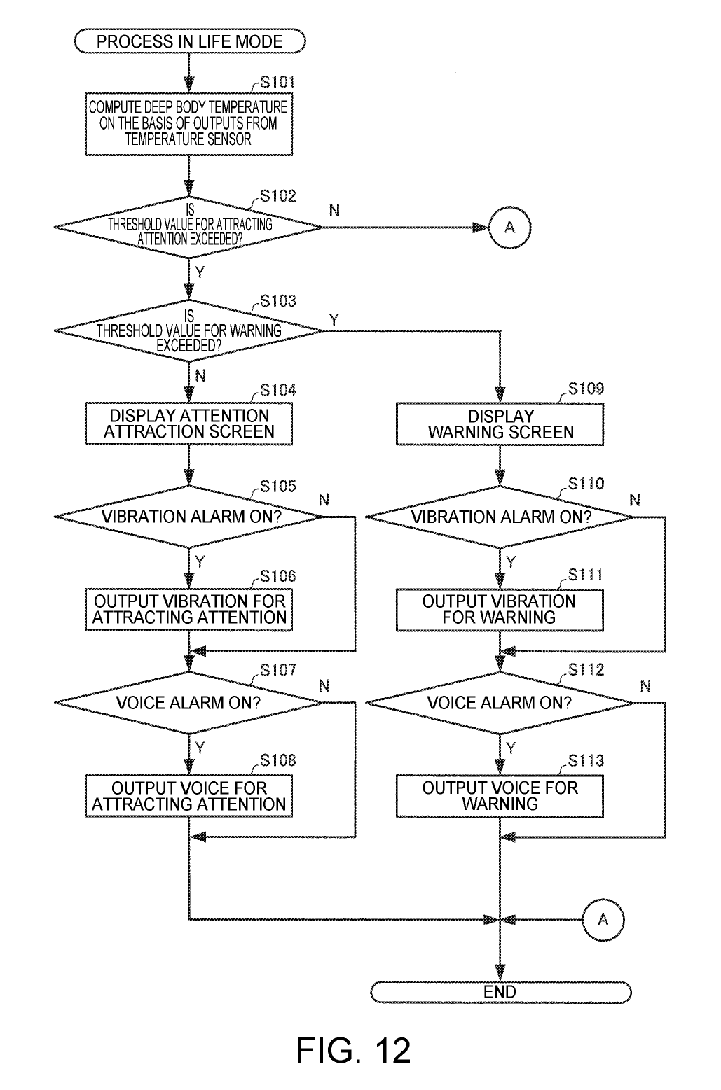

FIG. 12 illustrates an example of a flowchart of a process in the daily life mode. The flowchart is repeatedly executed by the processing section 120 of the electronic apparatus 1 during a period in which the electronic apparatus 1 is set to the daily life mode. A frequency of execution is a predetermined frequency such as 1/60 seconds.

If the flow is started, the processing section 120 computes a deep body temperature on the basis of outputs (a first temperature, a second temperature, and a third temperature) from the temperature sensor 116 (S101).

Next, the processing section 120 compares the deep body temperature with a threshold value for attracting attention, and determines whether or not the deep body temperature exceeds the threshold value (S102). The processing section 120 proceeds to the next determination process (S103) in a case where the deep body temperature exceeds the threshold value (S102Y), and finishes the flow if otherwise (S102N).

Next, the processing section 120 compares the deep body temperature with a threshold value for warning, and determines whether or not the deep body temperature exceeds the threshold value (S103). The processing section 120 proceeds to a warning process (S109) in a case where the deep body temperature exceeds the threshold value (S103Y), and proceeds to an alert process (S104) if otherwise (S103N).

Next, the processing section 120 displays an alert screen on the display section 170 (S104).

Next, the processing section 120 determines whether or not vibration output (vibration alarm) is permitted (ON) in advance by the user (S105). It is assumed that information indicating that a vibration alarm is permitted is stored in the storage section 130. The processing section 120 proceeds to the next process (S106) if the processing section 120 refers to the storage section 130 and recognizes that the vibration alarm is permitted (S105Y), and skips the next process (S106) if otherwise (S105N).

Next, the processing section 120 causes the sound output section 180 to output vibration for attracting attention (S106).

Next, the processing section 120 determines whether or not voice output (voice alarm) is permitted (ON) in advance by the user (S107). It is assumed that information indicating that a voice alarm is permitted is stored in the storage section 130. The processing section 120 proceeds to the next process (S108) if the processing section 120 refers to the storage section 130 and recognizes that the voice alarm is permitted (S107Y), and skips the next process (S108) if otherwise (S107N), and then finishes the flow.

Next, the processing section 120 causes the sound output section 180 to output a voice for attracting attention (S108), and finishes the flow.

On the other hand, in a case where the deep body temperature exceeds the threshold value in step S103 (S103Y), the processing section 120 displays a warning screen on the display section 170 (step S109).

Next, the processing section 120 determines whether or not vibration output (vibration alarm) is permitted (ON) in advance by the user (S110). It is assumed that information indicating that a vibration alarm is permitted is stored in the storage section 130. The processing section 120 proceeds to the next process (S111) if the processing section 120 refers to the storage section 130 and recognizes that the vibration alarm is permitted (S110Y), and skips the next process (S111) if otherwise (S110N).

Next, the processing section 120 causes the sound output section 180 to output vibration for warning (S111).

Next, the processing section 120 determines whether or not voice output (voice alarm) is permitted (ON) in advance by the user (S112). It is assumed that information indicating that a voice alarm is permitted is stored in the storage section 130. The processing section 120 proceeds to the next process (S113) if the processing section 120 refers to the storage section 130 and recognizes that the voice alarm is permitted (S112Y), and skips the next process (S113) if otherwise (S112N), and then finishes the flow.

Next, the processing section 120 causes the sound output section 180 to output a voice for warning (S113), and finishes the flow.

In the above-described flow, the order of execution of some of the steps may be replaced with each other in an allowable range. Some of the steps in the flow may be omitted.

### 1-3-9. Flow of Starting Process in Training Mode

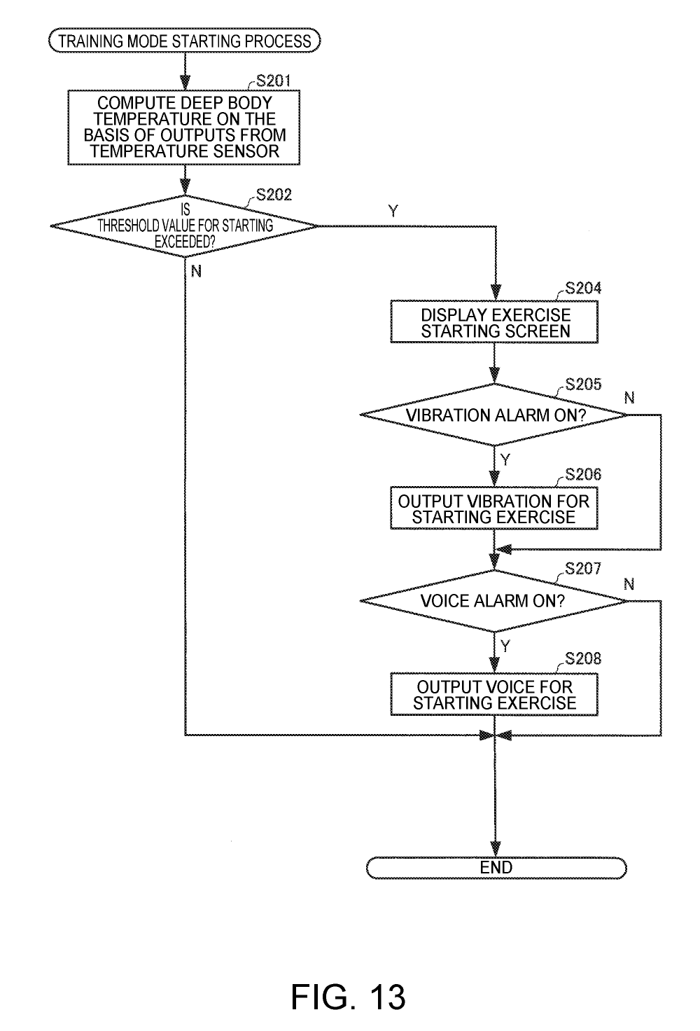

FIG. 13 illustrates an example of a flowchart of a starting process in the training mode. The flowchart is repeatedly executed by the processing section 120 of the electronic apparatus 1 during a period in which the electronic apparatus 1 is set to the training mode, and then an instruction for starting training is given. A frequency of execution is a predetermined frequency such as 1/60 seconds.

If the flow is started, the processing section 120 computes a deep body temperature on the basis of outputs (a first temperature, a second temperature, and a third temperature) from the temperature sensor 116 (S201). A computation process may be performed by the temperature sensor 116 (the same applies hereinafter).

Next, the processing section 120 compares the deep body temperature with a threshold value for starting, and determines whether or not the deep body temperature exceeds the threshold value (S202). The processing section 120 proceeds to the next process (S204) in a case where the deep body temperature exceeds the threshold value (S202Y), and finishes the flow if otherwise (S202N).

Next, the processing section 120 displays an exercise starting screen on the display section 170 (S204).

Next, the processing section 120 determines whether or not vibration output (vibration alarm) is permitted (ON) in advance by the user (S205). It is assumed that information indicating that a vibration alarm is permitted is stored in the storage section 130. The processing section 120 proceeds to the next process (S206) if the processing section 120 refers to the storage section 130 and recognizes that the vibration alarm is permitted (S205Y), and skips the next process (S206) if otherwise (S205N).

Next, the processing section 120 causes the sound output section 180 to output vibration for starting an exercise (S206).

Next, the processing section 120 determines whether or not voice output (voice alarm) is permitted (ON) in advance by the user (S207). It is assumed that information indicating that a voice alarm is permitted is stored in the storage section 130. The processing section 120 proceeds to the next process (S208) if the processing section 120 refers to the storage section 130 and recognizes that the voice alarm is permitted (S207Y), and skips the next process (S208) if otherwise (S207N), and then finishes the flow.

Next, the processing section 120 causes the sound output section 180 to output a voice for starting an exercise (S208), and finishes the flow.

In the above-described flow, the order of execution of some of the steps may be replaced with each other in an allowable range. Some of the steps in the flow may be omitted.

### 1-3-10. Flow of Stopping Process in Training Mode

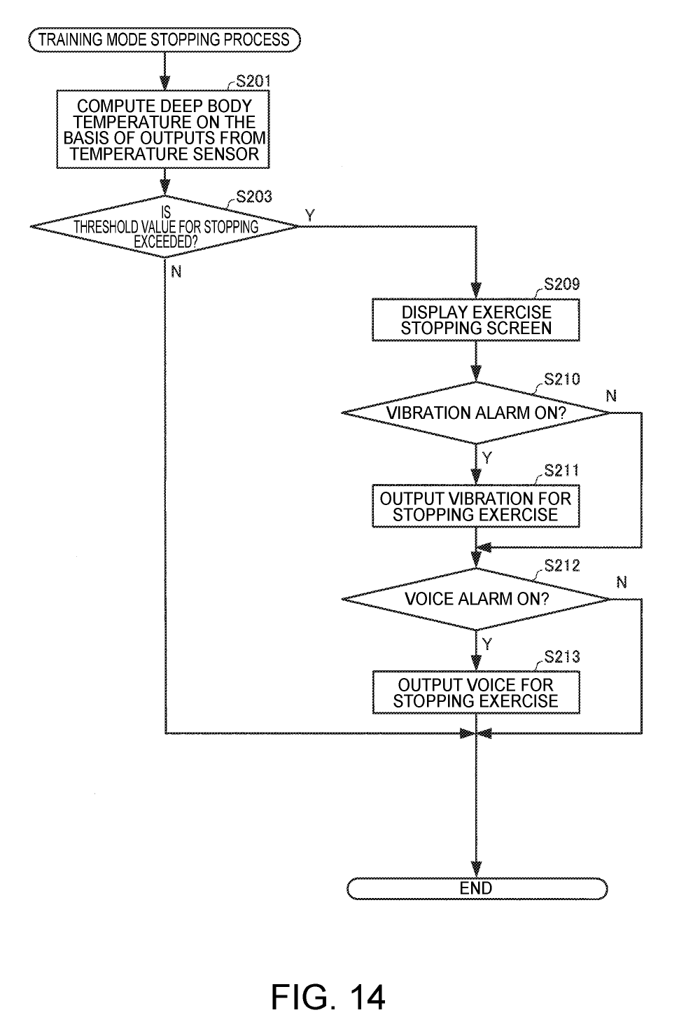

FIG. 14 illustrates an example of a flowchart of a stopping process in the training mode. The flowchart is repeatedly executed by the processing section 120 of the electronic apparatus 1 during a period in which an instruction for starting training is given, and then an instruction for stopping the training is given. A frequency of execution is a predetermined frequency such as 1/60 seconds.

If the flow is started, the processing section 120 computes a deep body temperature on the basis of outputs (a first temperature, a second temperature, and a third temperature) from the temperature sensor 116 (S201).

Next, the processing section 120 compares the deep body temperature with a threshold value for stopping, and determines whether or not the deep body temperature exceeds the threshold value (S203). The processing section 120 proceeds to the next process (S209) in a case where the deep body temperature exceeds the threshold value (S203Y), and finishes the flow if otherwise (S203N).

Next, the processing section 120 displays an exercise stopping screen on the display section 170 (S209).

Next, the processing section 120 determines whether or not vibration output (vibration alarm) is permitted (ON) in advance by the user (S210). It is assumed that information indicating that a vibration alarm is permitted is stored in the storage section 130. The processing section 120 proceeds to the next process (S211) if the processing section 120 refers to the storage section 130 and recognizes that the vibration alarm is permitted (S210Y), and skips the next process (S211) if otherwise (S210N).

Next, the processing section 120 causes the sound output section 180 to output vibration for stopping an exercise (S211).

Next, the processing section 120 determines whether or not voice output (voice alarm) is permitted (ON) in advance by the user (S212). It is assumed that information indicating that a voice alarm is permitted is stored in the storage section 130. The processing section 120 proceeds to the next process (S213) if the processing section 120 refers to the storage section 130 and recognizes that the voice alarm is permitted (S212Y), and skips the next process (S213) if otherwise (S212N), and then finishes the flow.

Next, the processing section 120 causes the sound output section 180 to output a voice for stopping an exercise (S213), and finishes the flow.

In the above-described flow, the order of execution of some of the steps may be replaced with each other in an allowable range. Some of the steps in the flow may be omitted.

### 1-3-11. Flow of Resuming Process in Training Mode

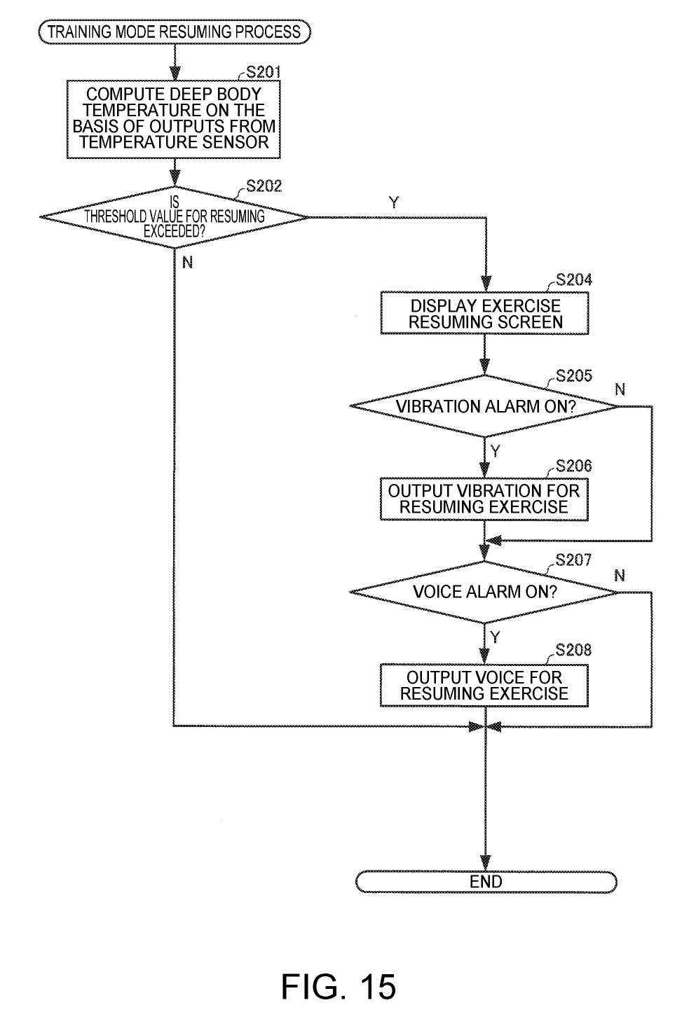

FIG. 15 illustrates an example of a flowchart of a resuming process in the training mode. The flowchart is repeatedly executed by the processing section 120 of the electronic apparatus 1 during a period in which an instruction for stopping training is given, and then an instruction for resuming the training is given. A frequency of execution is a predetermined frequency such as 1/60 seconds.

If the flow is started, the processing section 120 computes a deep body temperature on the basis of outputs (a first temperature, a second temperature, and a third temperature) from the temperature sensor 116 (S201).

Next, the processing section 120 compares the deep body temperature with a threshold value for resuming, and determines whether or not the deep body temperature exceeds the threshold value (S202). The processing section 120 proceeds to the next process (S204) in a case where the deep body temperature exceeds the threshold value (S202Y), and finishes the flow if otherwise (S202N).

Next, the processing section 120 displays an exercise resuming screen on the display section 170 (S204).

Next, the processing section 120 determines whether or not vibration output (vibration alarm) is permitted (ON) in advance by the user (S205). It is assumed that information indicating that a vibration alarm is permitted is stored in the storage section 130. The processing section 120 proceeds to the next process (S206) if the processing section 120 refers to the storage section 130 and recognizes that the vibration alarm is permitted (S205Y), and skips the next process (S206) if otherwise (S205N).

Next, the processing section 120 causes the sound output section 180 to output vibration for starting an exercise (S206).

Next, the processing section 120 determines whether or not voice output (voice alarm) is permitted (ON) in advance by the user (S207). It is assumed that information indicating that a voice alarm is permitted is stored in the storage section 130. The processing section 120 proceeds to the next process (S208) if the processing section 120 refers to the storage section 130 and recognizes that the voice alarm is permitted (S207Y), and skips the next process (S208) if otherwise (S207N), and then finishes the flow.

Next, the processing section 120 causes the sound output section 180 to output a voice for resuming an exercise (S208), and finishes the flow.

In the above-described flow, the order of execution of some of the steps may be replaced with each other in an allowable range. Some of the steps in the flow may be omitted.

### 2. Operations and Effects of Embodiment

As mentioned above, the processing section 120 of the electronic apparatus 1 measures a deep body temperature of a user, compares the deep body temperature with a predetermined comparison reference, and generates the above-described information regarding a physical condition on the basis of a result of the comparison. The display section 170 and the sound output section 180 of the electronic apparatus 1 presents the information to the user. The information is information based on a result of comparison with a predetermined comparison reference, and can thus be more effectively used for the life or training of a user than information itself indicating a deep body temperature. This is because an action to be performed by a user completely differs depending on a range in which a deep body temperature is included. Therefore, for example, the user may use the information as a reference when determining whether or not the content of the user's life is reconsidered or whether or not the content of the user's training is reconsidered.

Particularly, the electronic apparatus 1 in the daily life mode attracts a user's attention or warns the user about the risk of the user getting heatstroke during the life. Therefore, even if the user is not aware of a body temperature thereof or an ambient atmospheric temperature, in a case where the body of the user has fallen into a dangerous situation or is likely to fall thereinto, the user surely recognizes the situation, and can thus safely live the daily life. The electronic apparatus 1 gives advice corresponding to a deep body temperature of the user, and thus the user can perform an appropriate action such as water intake at a proper timing.

On the other hand, the electronic apparatus 1 in the training mode advises a user to start training in a case where a deep body temperature of the body of the user sufficiently increases (in a case where an oxygen intake reaches a state of being sufficiently high), advises the user to stop training in a case where a deep body temperature increases too much (in a case where the risk of heatstroke is high), and advises the user to resume training in a case where a deep body temperature is reduced too much. Therefore, the user can perform intensive training when a deep body temperature is within an appropriate range and can thus give a high load stimulus to the body thereof.

### 3. Modification Examples

### 3-1. Upload of Measured Data

In the system of the present embodiment, the processing section 120 of the electronic apparatus 1 preserves various pieces of measured data generated during the above-described measurement in the storage section 130 in a predetermined format as measured data along with date and time data indicating the date and time of the measurement. One or a plurality of pieces of measured data stored in the storage section 130 are transmitted to the information terminal 2 so as to be stored in the storage section 24 of the information terminal 2 as necessary. The measured data stored in the storage section 24 of the information terminal 2 is uploaded to the server 4 as necessary. The measured data uploaded to the server 4 is stored in the storage section 44 of the server 4. The measured data at various dates and times of various users stored in the storage section 44 of the server 4 are managed for each user for each date and time by the processing section 41 of the server 4. Communication between the electronic apparatus 1 and the information terminal 2 is performed via the communication section 190 of the electronic apparatus 1 and the communication section 22 of the information terminal 2, and communication between the information terminal 2 and the server 4 is performed via the communication section 27 of the information terminal 2, the network 3, and the communication section 42 of the server 4.

### 3-2. Variation of System Configuration

If the system of the present embodiment includes a portable apparatus having a sensor which can be mounted on a user's body, any one of the electronic apparatus 1, the sensor device 1C, the information terminal 2, and the server 4 may be omitted.

Sharing of functions in the system is not limited to the above description. For example, some or all of the functions of the sensor device 1C may be installed in the electronic apparatus 1, and may be installed in the information terminal 2. Some or all of the functions of the electronic apparatus 1 may be installed in the sensor device 1C, and may be installed in the information terminal 2. Some or all of the functions of the information terminal 2 may be installed in the sensor device 1C, and may be installed in the electronic apparatus 1. Some of the functions of the information terminal 2 may be installed in the server 4, and some of the functions of the server 4 may be installed in the information terminal 2.

### 3-3. Variation of Sensor

The sensor device 1C or the electronic apparatus 1 of the embodiment may use at least one of the following various sensors as a sensor. In other words, the various sensors are, for example, an acceleration sensor, a GPS (GNSS) sensor, an angular velocity sensor, a speed sensor, a heartbeat sensor (a chest belt or the like), a pulse sensor (a sensor performing measurement at locations other than the heart), a pedometer, a pressure sensor, an altitude sensor, a temperature sensor (an atmospheric temperature sensor or a body temperature sensor), a geomagnetic sensor, a weight meter (which is used as an external device of the system), an ultraviolet sensor, a perspiration sensor, a blood pressure sensor, a blood oxygen concentration (SpO₂) sensor, a lactic acid sensor, a blood sugar level sensor, and a wind speed sensor. A sensor which is not used for measurement may be not be mounted in the apparatus in the embodiment.

### 3-4. Notification Aspects

At least one of the electronic apparatus 1, the sensor device 1C, or the information terminal 2 may perform a notification of information for a user through image display, may perform a notification not only through image display but also through sound output or by using vibration, light, or a color (light emission from an LED or a display color of a display), and may perform a notification through a combination of at least two of image display, sound output, vibration, light, and a color.

### 3-5. Customizing

At least some of the notification content (including a notification period, a notification item, a notification aspect, a collecting method, a notification order, and the like) for a user in the electronic apparatus 1, the sensor device 1C, and the information terminal 2 of the embodiment may be set in advance by the user (customizable).

### 3-6. Forms of Apparatus

The electronic apparatus 1 may be configured as portable information terminals of various types, such as a wrist type electronic apparatus, an earphone type electronic apparatus, a ring type electronic apparatus, a pendant type electronic apparatus, an electronic apparatus attached to a sport appliance and used, a smart phone, a head mounted display (HMD), and a head up display (HUD). However, in a case where measured data obtained by the temperature sensor 116 is essential, a modification of the electronic apparatus 1 is performed within the scope of without obstructing measurement in the temperature sensor 116.

### 3-7. Optional Functions

Other functions may be installed in at least one of the sensor device 1C, the electronic apparatus 1, and the information terminal 2. Other functions may be, for example, well-known smart phone functions. The smart phone functions include, for example, a call function, a mail incoming notification function, a call incoming notification function, a communication function, and a camera function.

### 3-8. Positioning System

In the embodiment, as a satellite positioning system, a global positioning system (GPS) is used, but a global navigation satellite system (GNSS) may be used. For example, one or two or more of satellite positioning systems such as a European Geostationary-Satellite Navigation Overlay Service (EGNOS), a quasi zenith satellite system (QZSS), a global navigation satellite system (GLONASS), GALILEO, a BeiDou navigation satellite system (BeiDou) may be used. As at least one of the satellite positioning systems, a satellite-based augmentation system (SBAS) such as European geostationary-satellite navigation overlay service (EGNOS) or a wide area augmentation system (WAAS) may be used.

### 3-9. Example of HMD

Hereinafter, a description will be made of an example of a head mounted display with reference to FIG. 16.

A head mounted display 100 includes an image display section 20 (display section) which enables a user to visually recognize a virtual image in a state of being mounted on the head of the user, and a control device 10 which controls the image display section 20. The control device 10 also functions as a controller used for the user to operate the head mounted display 100. The image display section 20 is a mounting body which is mounted on the head of the user, and has a spectacle-shaped frame 2′ (main body) in the present embodiment.

As the control device 10, the information terminal 2 illustrated in FIG. 1 may be used. In this case, the operation section 23 of the information terminal 2 functions as a determination key 11, a display changing key 13, a track pad 14, a luminance changing key 15, a direction key 16, a menu key 17, and the like. FIG. 16 illustrates an example in which the image display section 20 and the control device 10 are connected to each other via a connection unit 40 (a main body cord 48′, a right cord 42′, a left cord 44′, and a cable 48), but the image display section 20 and the control device 10 may be connected to each other through wireless communication.

The frame 2′ has a right holding unit 21′ and a left holding unit 23′. The right holding unit 21′ is a member which is provided so as to extend over a position corresponding to the temporal region of the user from an end part ER which is the other end of a right optical image display unit 26′ when the user wears the image display section 20. Similarly, the left holding unit 23 is a member which is provided so as to extend over a position corresponding to the temporal region of the user from an end part EL which is the other end of a left optical image display unit 28′ when the user wears the image display section 20. The right holding unit 23′ comes into contact with the right ear or the vicinity thereof on the head of the user, and the left holding unit 23′ comes into contact with the left ear or the vicinity thereof so that the right holding unit 21′ and the left holding unit 23′ hold the image display section 20 on the head of the user. The right holding unit 21′ and the left holding unit 23′ hold the image display section 20 on the head of the user in the same manner as temples of spectacles.

In the present embodiment, the spectacle type frame 2′ will be described as an example of the main body. A shape of the main body is not limited to a spectacle shape, and may be any shape as long as the main body is mounted on and fixed to the head of the user, and is more preferably a shape which causes the main body to be hung in front of both the eyes of the user. For example, in addition to the spectacle shape described here, a shape of the main body may be a snow goggle shape covering the upper part of the face of the user, and may be a shape which is disposed in front of each of the right and left eyes of the user, such as binoculars.

The frame 2′ is provided with a right display driving unit 22′, a left display driving unit 24′, the right optical image display unit 26′, the left optical image display unit 28′, and a microphone 63. The right display driving unit 22′ and the left display driving unit 24′ are disposed on a side opposing the head of the user when the user wears the image display section 20. The right optical image display unit 26′ and the left optical image display unit 28′ are respectively located in front of the right eye and the left eye of the user when the user wears the image display section 20. One end of the right optical image display unit 26′ and one end of the left optical image display unit 28′ are connected to each other at the position corresponding to the glabella of the user when the user wears the image display section 20.

### 4. Others

The invention is not limited to the above-described embodiment, and may be variously modified within the scope of the spirit of the invention.

The above-described embodiment and modification examples are only examples, and the invention is not limited thereto. For example, the embodiment and the respective modification examples may be combined with each other as appropriate.

The invention includes substantially the same configuration (for example, a configuration in which functions, methods, and results are the same, or a configuration in which objects and effects are the same) as the configuration described in the embodiment. The invention includes a configuration in which an inessential part of the configuration described in the embodiment is replaced with another part. The invention includes a configuration which achieves the same operation and effect or a configuration capable of achieving the same object as in the configuration described in the embodiment. The invention includes a configuration in which a well-known technique is added to the configuration described in the embodiment.

What is claimed is:

1. A living body monitoring system comprising:
a portable electronic apparatus including
a measurement unit that measures a deep body temperature of a user's body, and
a processor that compares the deep body temperature with a predetermined reference, and generates information regarding a physical condition of the user on the basis of a result of the comparison; and
an information terminal including
a communication unit that receives the information regarding a physical condition of the user from the portable electronic apparatus, and
a display that presents the information to the user.

2. The living body monitoring system according to claim 1,
wherein the information regarding the physical condition includes advice to the user.

3. The living body monitoring system according to claim 2,
wherein the advice includes at least one of advice related to whether or not starting of an exercise is possible and advice related to whether or not an exercise is required to be stopped.

4. The living body monitoring system according to claim 3,
wherein the advice includes at least one of advice related to the necessity of water intake, advice related to the necessity of rest, advice related to the necessity of atmospheric temperature adjustment, advice related to the necessity of body temperature adjustment, and advice related to the necessity of doctor's examination.

5. The living body monitoring system according to claim 1,
wherein the information regarding the physical condition includes at least one of information regarding a point where water intake is possible, information regarding a point where a rest is possible, information regarding a point where atmospheric temperature adjustment is possible, information regarding a point where body temperature adjustment is possible, and information regarding a point where a doctor's examination is possible.

6. The living body monitoring system according to claim 1,
wherein the information regarding the physical condition is information which can be recognized by the user through at least one of an auditory sense, a visual sense, and a tactile sense.

7. The living body monitoring system according to claim 1,
wherein the information regarding the physical condition is displayed by using at least one of a color, text, a symbol, a scale, and a pattern which can be recognized through a visual sense by the user.

8. A portable electronic apparatus comprising:
a temperature sensor that measures a deep body temperature of a user's body;
at least one processing section that compares the deep body temperature with a predetermined comparison reference, and generates information regarding a physical condition of the user on the basis of a result of the comparison; and
a display that presents the information to the user.

9. The portable electronic apparatus according to claim 8,
wherein the information regarding the physical condition includes at least one of advice related to whether or not starting of an exercise is possible and advice related to whether or not an exercise is required to be stopped.

10. The portable electronic apparatus according to claim 8,
wherein the information regarding the physical condition includes at least one of advice related to the necessity of water intake, advice related to the necessity of rest, advice related to the necessity of atmospheric temperature adjustment, advice related to the necessity of body temperature adjustment, and advice related to the necessity of doctor's examination.

11. A computer readable recording medium recording a living body monitoring program causing a computer to execute:
measuring a deep body temperature of a user's body;
comparing the deep body temperature with a predetermined comparison reference;
generating information regarding a physical condition of the user on the basis of a result of the comparison; and
presenting the information to the user.

12. A living body monitoring method comprising:
measuring a deep body temperature of a user's body;
comparing the deep body temperature with a predetermined comparison reference;
generating information regarding a physical condition of the user on the basis of a result of the comparison; and
presenting the information to the user.

13. The living body monitoring method according to claim 12,
wherein the information regarding the physical condition includes advice to the user.

14. The living body monitoring method according to claim 13,
wherein the advice includes at least one of advice related to whether or not starting of an exercise is possible and advice related to whether or not an exercise is required to be stopped.

15. The living body monitoring method according to claim 13,
wherein the advice includes at least one of advice related to the necessity of water intake, advice related to the necessity of rest, advice related to the necessity of atmospheric temperature adjustment, advice related to the necessity of body temperature adjustment, and advice related to the necessity of doctor's examination.

16. The living body monitoring method according to claim 12,
wherein the information regarding the physical condition includes at least one of a point where water intake is possible, information regarding a point where a rest is possible, information regarding a point where atmospheric temperature adjustment is possible, information regarding a point where body temperature adjustment is possible, and information regarding a point where a doctor's examination is possible.

17. The living body monitoring method according to claim 12,
wherein the information regarding the physical condition is information which can be recognized by the user through at least one of an auditory sense, a visual sense, and a tactile sense.

18. The living body monitoring method according to claim 12,
wherein the information regarding the physical condition is displayed by using at least one of a color, text, a symbol, a scale, and a pattern which can be recognized through a visual sense by the user.
